(19) United States
(12) Patent Application Publication
(10) Pub. No.: US 2022/0090142 A1
(43) Pub. Date: Mar. 24, 2022

(54) TRANSPOSASE WITH ENHANCED INSERTION SITE SELECTION PROPERTIES

(71) Applicant: Probiogen AG, Berlin (DE)

(72) Inventors: Sven Krügener, Berlin (DE); Thomas Rose, Blankenfelde (DE); Volker Sandig, Berlin (DE); Karsten Winkler, Berlin (DE)

(21) Appl. No.: 17/420,076

(22) PCT Filed: Feb. 13, 2019
(86) PCT No.: PCT/EP2019/053571

§ 371 Date: Jun. 30, 2021

(51) Int. Cl. C12N 15/90; C12N 15/85; C12N 9/10

(52) U.S. Cl. CPC C12N 15/90; C12N 15/85; C12N 2800/90; C12Y 203/01048; C07K 2319/80; C12N 9/1029

(57) ABSTRACT

The present invention relates to a polypeptide comprising a transposase and at least one heterologous chromatin reader element (CRE). Further, the present invention relates to a polynucleotide encoding the polypeptide. Furthermore, the present invention relates to a vector comprising the polynucleotide. In addition, the present invention relates to a kit comprising a transposase and at least one heterologous chromatin reader element (CRE).

The present invention relates to a polypeptide comprising a transposase and at least one heterologous chromatin reader element (CRE). Further, the present invention relates to a polynucleotide encoding the polypeptide. Furthermore, the present invention relates to a vector comprising the polynucleotide. In addition, the present invention relates to a kit comprising a transposase and at least one heterologous chromatin reader element (CRE).

### BACKGROUND OF THE INVENTION

Transposons have recently been developed as potent, non-viral gene delivery tools. In particular, the performance of a generated producer cell line can be improved, when the integration of plasmid DNA is supported using a transposon. For instance, a transposon allows the integration of a greater size of heterologous DNA and the integration of a higher number of heterologous DNA copies into each genome. Furthermore, integration via a transposon provides an efficient method for the reduction of plasmid backbone integration and/or the reduction of concatemers.

Transposable elements or transposons are DNA-sections, which can move from one locus to another part of the genome. Two classes of transposable elements are distinguished: retrotransposons, which replicate through an RNA intermediate (class 1), and “cut-and-paste” DNA transposons (class 2). Class 2 transposons are characterised by short inverted terminal repeats (ITRs) and element-encoded transposases, enzymes with excision and insertion activity. 23 superfamilies of DNA transposons are currently described [Bao et al., 2015 [doi: 10.1186/s13100-015-0041-9.]]. In the natural configuration, the transposase gene is located between the inverted repeats. A number of class 2 transposons have been shown to facilitate insertion of heterologous DNA into the genome of eukaryotes, for example, a transposon from the moth Trichoplusia ni (PiggyBac), a transposon from the bat Myotis lucifugus (PiggyBat), a reconstructed transposon from salmon species (Sleeping Beauty), or a transposon from the medaka Oryzias latipes (Tol2). These transposons have many applications in genetic manipulation of a host genome, including transgene delivery and insertional mutagenesis. For instance, the PiggyBac (PB) DNA transposon (previously described as IFP2) is used technologically and commercially in genetic engineering by virtue of its property to efficiently transpose between vectors and chromosomes [U.S. Pat. No. 6,218,185 B1]. For these applications the DNA to be integrated is flanked by two PB ITRs in a PB vector. By co-delivery of PB transposase the flanked DNA is excised precisely form the PB vector and integrated into the target genome at TTAA specific sites.

The genomic integration site preferences of transposable elements vary between different superfamilies. For instance, transposable elements of the PiggyBac superfamily (e.g. PiggyBac and PiggyBat) are enriched at transcriptional units, CpG islands, and transcriptional start sites (TSSs) and are co-localized with BRD4 binding sites found predominately in the proximity of differentiation induced genes (Gogol-Döring et al., 2016 doi: [10.1038/mt.2016.11], Galvan et al., 2009 doi: [10.1097/CJI.0b013e3181b2914c]). Since host cell factors are involved in integration, efficiency of PiggyBac transposases can vary substantially among cell lines.

To increase transformation efficiencies, more active transposases were developed. These hyperactive transposases yield a greater fraction of cells that integrated a provided transposon and a greater number of transposon integrations per cell compared to wild-type transposases. Different strategies are described in the art: For example, U.S. Pat. No. 8,399,643 B2 describes hyperactive PiggyBac transposases and EP2160461B1 describes hyperactive Sleeping Beauty transposases generated via side directed mutagenesis, U.S. Pat. No. 9,534,234 B2 provides a PiggyBac-like transposase derived from the silkworm Bombyx mori and from the frog Xenopus tropicalis fused to a heterologous nuclear localization sequence (NLS), EP1546322 B1 discloses a chimeric integrating enzyme comprising a binding domain recognising a DNA landing pad to drag transposon-transposase complex to the landing pad and promote integration in its vinicity and EP1594972B1 claims a transposase or a fragment or derivative thereof having transposase function fused to a polypeptide binding domain that can associates with a cellular or engineered polypeptide comprising a DNA targeting domain.

Furthermore, excision competent but integration defective PiggyBac transpoases were generated via side directed mutagenesis, to avoid further genome modification following PiggyBac excision by reintegration (U.S. Pat. No. 9,670,503 B2).

The hyperactive transposases described in the art show increased excision and/or integration activity of the transposase or they support the import of the transposon-transposase complex into the cell nucleus by fusing heterologous nuclear localization sequences (NLS). Some of the described transposases support the docking of the transposon-transposase complex to a specific site of the host genome by fusing specific DNA binding domains. These site-specific transposases allow the defined integration of transposons at known or previously inserted landing pads in the respective cell line. With this modification, the transposases can be applied in a similar fashion as site specific recombinases such as cre and flp. However, in contrast to the above-mentioned recombinases, integration occurs in the vicinity of the site but not at the exact position of the selected site providing no clear advantage over recombinases. In addition, the integration site does not necessarily have to be located in transcriptionally active chromosomal regions resulting in low product yields.

Based on the above, it would be highly desirable to direct genes to random positions with high transcriptional activity, in particular to generate producer cell lines for the production of therapeutic proteins or for the production of biopharmaceutical products based on virus particles in high yields.

Besides methylation of the DNA itself, chemical modifications of histones are involved in the epigenetic regulation of gene expression. While methylation of CpG dinucleotides is stably maintained not only within cell lineages and but also inherited through generations, histone modifications are intertwined with DNA methylation but generally more short lived. A large number of different post-translational modifications (PTMs) of histones are discovered and the recruitment of specific proteins and protein complexes by histone marks is now an accepted dogma of how histone modifications mediate their function. Histone modifications can influence transcription and affect other DNA processes such as replication, recombination, and repair.

Histone methylation mainly occurs on the side chains of arginine and lysine. Arginine may be mono-, symmetrically or asymmetrically di-methylated, whereas lysine may be mono-, di- or tri-methylated. While some methylation states are associated with enhanced expression others cause repression. A trimethylated lysine 4 on the histone H3 protein (H3K4me3) is typically found at promoters of actively described genes.

Acetylation of lysine is highly dynamic and regulated by histone acetyltransferases and histone deacetylases in response to various stimuli. The positive charge on a histone is removed by acetylation, by which the interaction of the N-termini of the histone with the negatively charged phosphate groups of the DNA is decreased, which in turn is associated with greater levels of transcription of nearby genes. Histone modifying enzymes act in concert and are well balanced. In cancer cells and transformed cell lines this balance is disturbed, in particular that of parental histone recycling and de novo assembly.

Chromatin reader proteins bind to histone tails recognising specific PTMs to recruit chromatin remodelling complexes and components of the transcriptional machinery. For example, bromodomains found in chromatin-associated proteins like histone acetyltransferases specifically recognise acetylated lysine residues and plant homeodomain (PHD) zinc fingers of other chromatin-associated proteins bind to H3K4me3. In contrast to CpG islands that tend to be associated with active genes in general, the described histone modifications provide short-term epigenetic memory and may be reversed after a few cell divisions, in particular in transformed cell lines.

As mentioned above, it would be highly desirable to direct genes to random positions with high transcriptional activity, in particular to generate producer cell lines for the production of therapeutic proteins or for the production of biopharmaceutical products based on virus particles in high yields.

Transposons or transposases that recognise specific post-translational histone modifications (methylations and/or acetylations) are not described or suggested in art. It was unlikely that such targeting has any effect at all if histones have to be displaced for transposition to occur. Moreover, it was likely that the transposition itself would disturb histone modifications.

The present inventors surprisingly found that an artificial transposable element comprising at least one polynucleotide of interest can effectively be targeted to active chromatin via a transposase coupled with at least one heterologous chromatin reader element. The present inventors surprisingly established, for the first time, a targeting system comprising an artificial transposable element comprising at least one polynucleotide of interest and a polypeptide comprising a transposase coupled with at least one heterologous chromatin reader element for the production of proteins and viruses in high yields. The present inventors found that the higher protein levels were not the result of higher transgene copy number but the result of efficient transgene integration into highly active genomic loci.

### SUMMARY OF THE INVENTION

In a first aspect, the present invention relates to a polypeptide comprising a transposase or a fragment or a derivative thereof having transposase function and at least one heterologous chromatin reader element (CRE).

In a second aspect, the present invention relates to a polynucleotide encoding the polypeptide according to the first aspect.

In a third aspect, the present invention relates to a vector comprising the polynucleotide according to the second aspect.

In a fourth aspect, the present invention relates to a method for producing a transgenic cell comprising the steps of:
(i) providing a cell, and
(ii) introducing
   a transposable element comprising at least one polynucleotide of interest, and
   a polypeptide according to the first aspect,
   a polynucleotide according to the second aspect, or
   a vector according to the third aspect
   into the cell, thereby producing/obtaining the transgenic cell.

In a fifth aspect, the present invention relates to a transgenic cell obtainable by the method according to the fourth aspect.

In a sixth aspect, the present invention relates to the use of a transgenic cell according to the fifth aspect for the production of a protein or virus.

In a seventh aspect, the present invention relates to a kit comprising
(i) a transposable element comprising a cloning site for inserting at least one polynucleotide of interest, and
(ii) a polypeptide according to the first aspect,
   a polynucleotide according to the second aspect,
   a vector according to the third aspect, or
   at least one heterologous CRE and a polypeptide comprising a transposase or a fragment or a derivative thereof having transposase function.

In an eight aspect, the present invention relates to a targeting system comprising
(i) a transposable element comprising at least one polynucleotide of interest, and a polypeptide according to the first aspect,
(ii) a transposable element comprising at least one polynucleotide of interest, and a polynucleotide according to the second aspect,
(iii) a transposable element comprising at least one polynucleotide of interest, and a vector according to the third aspect,
(iv) a transposable element comprising at least one polynucleotide of interest,
   at least one heterologous CRE associated with the transposable element, and
   a polypeptide comprising a transposase or a fragment or a derivative thereof having transposase function.

This summary of the invention does not necessarily describe all features of the present invention. Other embodiments will become apparent from a review of the ensuing detailed description.

### DETAILED DESCRIPTION OF THE INVENTION

### Definitions

Before the present invention is described in detail below, it is to be understood that this invention is not limited to the particular methodology, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

Preferably, the terms used herein are defined as described in “A multilingual glossary of biotechnological terms: (IUPAC Recommendations)”, Leuenberger, H. G. W, Nagel, B. and Kölbl, H. eds. (1995), Helvetica Chimica Acta, CH-4010 Basel, Switzerland).

Several documents are cited throughout the text of this specification. Each of the documents cited herein (including all patents, patent applications, scientific publications, manufacturer's specifications, instructions, GenBank Accession Number sequence submissions etc.), whether supra or infra, is hereby incorporated by reference in its entirety. Nothing herein is to be construed as an admission that the invention is not entitled to antedate such disclosure by virtue of prior invention. In the event of a conflict between the definitions or teachings of such incorporated references and definitions or teachings recited in the present specification, the text of the present specification takes precedence.

The term “comprise” or variations such as “comprises” or “comprising” according to the present invention means the inclusion of a stated integer or group of integers but not the exclusion of any other integer or group of integers. The term “consisting essentially of” according to the present invention means the inclusion of a stated integer or group of integers, while excluding modifications or other integers which would materially affect or alter the stated integer. The term “consisting of” or variations such as “consists of” according to the present invention means the inclusion of a stated integer or group of integers and the exclusion of any other integer or group of integers.

The terms “a” and “an” and “the” and similar reference used in the context of describing the invention (especially in the context of the claims) are to be construed to cover both the singular and the plural, unless otherwise indicated herein or clearly contradicted by context.

The term “chromatin”, as used herein, refers to a complex of DNA and protein found in cells, in particular eukaryotic cells. The primary function of chromatin is packaging and folding DNA molecules into a more compact, denser shape. This prevents the DNA molecules from becoming tangled and plays important roles in reinforcing the DNA during cell division, preventing DNA damage, and regulation gene expression and DNA replication. The primary protein components of chromatin are histones which bind to DNA and function as so called “anchors” around which the DNA strands are wound. In general, there are three levels of chromatin organization: (i) DNA wraps around histone proteins, forming nucleosomes and the so-called “beads on a string” structure (euchromatin), (ii) multiple histones wrap into a 30-nanometer fiber consisting of nucleosome arrays in their most compact form (heterochromatin), and (iii) higher-level DNA supercoiling of the 30-nm fiber produces the metaphase chromosome (during mitosis and meiosis). Formation of higher order chromatin not only results in condensing DNA, but also affects its functionality since certain regions of DNA are no longer accessible whereas some other regions will be more accessible for, e.g. effector proteins or components of the transcriptional machinery to bind.

The term “histones”, as used herein, refers to the building blocks of chromatin. Histones are small basic tripartite proteins that are composed of a globular domain and unstructured N- or C-terminal tails. Histones can be covalently modified by methylation (e.g. lysine methylation or arginine methylation), acetylation, phosphorylation, and/or ubiquitination at their flexible N- or C-terminal tails as well as at their globular domains. Post-translational modifications (PTMs) of histones are key players in the regulation of chromatin function. While euchromatin, represents the transcriptionally active, loosely packaged and gene-rich region chromatin, heterochromatin represents the highly condensed and gene-poor chromatin. The transition between euchromatin and heterochromatin is largely influenced by mechanisms involving DNA methylation, non-coding RNAs and RNA interference (RNAi), DNA replication-independent incorporation of histone variants and histone post-translational modifications (PTMs).

As suggested by the “histone code hypothesis”, distributions of histone PTMs form a signature that is indicative of the chromatin state of a given loci. Euchromatin is generally associated with high levels of histone acetylation and/or methylation, in particular mono-methylation. In particular, acetylation, e.g. of lysine residues, can reduce the positive charge of histones, thereby weakening their interaction with negatively charged DNA and increasing nucleosome (complex of DNA and histone) fluidity. Also amino acid acetylation can reduce the compaction level of a nucleosomal array. The chromatin state of a given loci depends, for example, on molecules which can posttranslationally modify, e.g. methylate and/or acetylate, histones (so called “writers”), molecules which can remove posttranslational modifications, e.g. methylated and/or acetylated histones (so called “erasers”), and molecules, which can readily identify posttranslational modifications of histones, e.g. methylations and/or acetylations, (so called “readers”). The “reader” molecules are recruited to such histone modifications and bind via specific domains, e.g. plant homeodomain (PHD) zinc finger, bromodomain, or chromodomain. The triple action of “writing”, “reading”, and “erasing” establishes the favourable local environment for transcriptional regulation, DNA damage repair, etc.

The term “chromatin reader element (CRE)”, as used herein, refers to any structure providing an accessible surface (such as a cavity or surface groove) to accommodate a modified histone residue and determine the type of post-translational histone modification (e.g. acetylation or methylation and acetylation versus methylation) or state specificity (such as mono-methylation, di-methylation, versus tri-methylation, e.g. of lysines or arginines). A “chromatin reader element” also interacts with the flanking sequence of the modified amino acid in order to distinguish sequence context. In particular, a “chromatin reader element” binds histone tails and recognizes specific post-translational modifications (PTMs), e.g. methylations, such as lysine or arginine methylations, and/or acetylations, on the histones. As a consequence, the chromatin reader element recruits chromatin remodelling complexes and components of the transcriptional machinery to the binding position. The “chromatin reader element” is preferably an element recognizing the histone methylation degree, in particular histone mono-methylation, di-methylation or, tri-methylation degree, e.g. of lysine and/or arginine residues. Alternatively, the “chromatin reader element” is an element recognizing the acetylation state of histones. As mentioned above, transcriptionally active euchromatin is generally associated with histone acetylation and/or methylation, in particular histone mono-methylation. It is preferred that the the chromatin reader element is a “chromatin reader domain (CRD)”. The chromatin reader domain may be a bromodomain, a chromodomain, a plant homeodomain (PHD) zinc finger, a WD40 domain, a tudor domain, double/tandem tudor domain, a MBT domain, an ankyrin repeat domain, a zf-CW domain, or a PWWP domain. For example, bromodomains are found in chromatin-associated proteins like histone acetyltransferases specifically recognizing acetylated lysine residues. PHDs (in particular PHD fingers) are also found in chromatin-associated proteins like plant homeodomain proteins such as transcription initiation factors. They can also recognize acetylated lysine residues. Chromatin reader domains that recognize histone methylation include PHD domains, chromodomains, WD40 domains, tudor domains, double/tandem tudor domains, MBT domains, ankyrin repeat domains, zf-CW domains, and PWWP domains. It is more preferred that the chromatin reader domain is a bromodomain or a plant homeodomain (PHD) zinc finger. It is alternatively preferred that the chromatin reader element is an artificial chromatin reader element. The artificial chromatin reader element may be a micro antibody, a single chain antibody, an antibody fragment, an affibody, an affilin, an anticalin, an atrimer, a DARPin, a FN2 scaffold, a fynomer, or a Kunitz domain. In this respect, the term “micro antibody”, as used herein, refers to an artificial short chain of amino acids copied from a fully functional natural antibody.

The term “antibody fragment”, as used in the context of the present invention, refers to a fragment of an antibody that contains at least domains capable of specific binding to an antigen, i.e. chains of at least one V_{L} and/or V_{H}-domain or binding part thereof.

In the context of the present invention, the chromatin reader element, in particular chromatin reader domain, is associated with a transposase, or a fragment, or a derivative thereof having transposase function. The transposase, or a fragment, or a derivative thereof having transposase function connected to a chromatin reader element, in particular chromatin reader domain, is able to recognize specific histone post-translational modifications, such as methylations and/or acetylations and, thus, active euchromatin.

The term “transposase”, as used herein, refers to any enzyme that is able to bind to the ends of a transposable element and to catalyze its movement to another part of the genome by a cut and paste mechanism or a replicative transposition mechanism. The ends of a transposable element are preferably terminal repeats, e.g. inverted terminal repeats (ITRs) or long terminal repeats (LTRs). Thus, a transposase is not only able to recognize the terminal repeats surrounding the mobile element, it is also able to recognize target sequences, e.g. on the new host DNA.

The term “fragment” of a transposase “having transposase function” refers to a fragment derived from a naturally occurring transposase which lacks one or more amino acids compared to the naturally occurring transposase and has transposase function. For example, said fragment of a naturally occurring transposase has still transposase function, in particular still mediates nucleotide sequence, e.g. DNA, excision and/or insertion, or has an improved transposase function, in particular an improved activity/ability to mediate nucleotide sequence, e.g. DNA, excision and/or insertion. Generally, a fragment of an amino acid sequence contains less amino acids than the corresponding full length sequence, wherein the amino acid sequence present is in the same consecutive order as in the full length sequence. As such, a fragment does not contain internal insertions or deletions of anything into the portion of the full length sequence represented by the fragment.

The term “derivative” of a transposase “having transposase function” refers to a derivative of a naturally occurring transposase, wherein one or more amino acids have been substituted, deleted, and/or added compared to the naturally occurring transposase and has transposase function. For example, said derivative of a naturally occurring transposase has still transposase function, in particular still mediates nucleotide sequence, e.g. DNA, excision and/or insertion, or has an improved transposase function, in particular an improved activity/ability to mediate nucleotide sequence, e.g. DNA, excision and/or insertion. In contrast to a fragment, a derivative may contain internal insertions or deletions within the amino acids that correspond to the full length sequence, or may have similarity to the full length coding sequence.

The above described modifications are preferably effected by recombinant DNA technology. Further modifications may also be effected by applying chemical alterations to the transposase.

The transposase (as well as fragments or derivatives thereof) may be recombinantly produced and yet may retain identical or essentially identical features as the naturally occurring transposase, in particular with respect to nucleotide sequence, e.g. DNA, excision and/or insertion. For example, the transposase fragment or derivative referred to herein preferably maintain at least 50% of the activity of the native protein, more preferably at least 75%, and even more preferably at least 95% of the activity of the native protein. Such biological activity is readily determined by a number of assays known in the art, for example, enzyme activity assays. Alternatively, the transposase (as well as fragments or derivatives thereof) may be recombinantly produced and yet may have improved features compared to the naturally occurring transposase, in particular with respect to nucleotide sequence, e.g. DNA, excision and/or insertion. For example, the transposase fragment or derivative referred to herein preferably have an activity which is at least 20% above the activity of the native protein, more preferably at least 50%, and even more preferably at least 75% above of the activity of the native protein. Such biological activity is readily determined by a number of assays known in the art, for example, enzyme activity assays.

The transposase or fragment or derivative thereof having transposase function may be a recombinant, an artificial, and/or a heterologous transposase or fragment or derivative thereof having transposase function.

The transposase may be a transposase of class I (retrotransposase) or a transposase of class II (DNA transposase). In case of a transposase of class I, the transposase may also be designated as integrase.

The term “transposable element” (also designated as “transposon” or “jumping gene”), as used herein, refers to a polynucleotide molecule that can change its position within the genome. Usually, the transposable element includes a polynucleotide encoding a functional transposase that catalyses excision and insertion. However, the transposable element described in the context of the present invention is devoid of a polynucleotide encoding a functional transposase. The transposon based polynucleotide molecule described herein no longer comprises the complete sequence encoding a functional, preferably a naturally occurring, transposase. Preferably, the complete sequence encoding a functional, preferably a naturally occurring, transposase or a portion thereof, is deleted from the transposable element. Alternatively, the gene encoding the transposase is mutated such that a naturally occurring transposase or a fragment or derivative thereof having the function of a transposase, i.e. mediating the excision and/or insertion of a transposon into a target site, is no longer contained.

The transposable element described herein retains sequences that are required for mobilization by the transposase provided in trans. These are the repetitive sequences at each end of the transposable element containing the binding sites for the transposase allowing the excision and integration. Said repetitive sequences are also called terminal repeats. Preferably, the terminal repeats are inverted terminal repeats (ITRs) or long terminal repeats (LTRs).
Instead of polynucleotide sequences encoding a functional transposase, exogenous polynucleotide sequences, e.g. polynucleotide sequences of interest/heterologous polynucleotide sequences such as functional genes and regulatory elements driving expression, are part of the transposable element described herein. Thus, said transposable element may also be designated as recombinant/artificial transposable element.
The transposable element may be derived from a bacterial or a eukaryotic transposable element wherein the latter is preferred. Further, the transposable element may be derived from a class I or class II transposable element. Class II or DNA-based transposable elements are preferred for gene transfer applications, because transposition of these elements does not involve a reverse transcription step (involved in transposition of Class 1 or retrotransposable elements).
Class II or DNA-based transposable elements contain inverted terminal repeats (ITRs) at either end. Conservative DNA-based transposable elements move by a cut-and-paste mechanism. This requires a transposase, inverted repeats at the ends of the transposable element and a target sequence on the new host DNA molecule. As described above, the transposase is provided in the present invention in trans. In the cut-and-paste mechanism, the transposase binds to the inverted terminal repeats of the transposable element and cuts the transposable element out of the current location. The transposase then locates the target sequence, cuts the DNA backbone in staggered location, which leaves a slight single-stranded overhang on the new host DNA molecule and then inserts the transposable element. The transposable element does not completely fill the single-stranded pieces of DNA. The host organism, e.g. host cell, recognizes the short, single, stranded DNA segments and fills in the gaps. This process is called conservative transposition and leaves the transposable element unaltered. During the removal of the transposon, the original DNA suffers a double-stranded break that usually dooms this molecule. Therefore, transposition is tightly regulated.
Preferably, the transposase recognises a TA dinucleotide at each end of the transposable element, particularly at the repetitive sequences of the transposable element and excises the transposable element, e.g. from a vector. Usually, two transposase monomers are involved in the excision of the transposable element, one transposase monomer at each end of the transposable element. Finally, the transposase dimer in complex with the excised transposable element reintegrates the transposable element in the DNA of a host organism, e.g. host cell, by recognising a TA dinucleotide in the target sequence.
The transposable element may be a recombinant, an artificial, and/or a heterologous transposable element.

The present inventors found that said (recombinant/artificial) transposable element in combination with a polypeptide comprising a transposase and at least one chromatin reader element allows the targeting of the transposable element to random positions in the genome with high transcriptional activity. In other words, the present inventors found that said (recombinant/artificial) transposable element in combination with a polypeptide comprising a transposase and at least one chromatin reader domain allows the targeting of active chromatin. The result of this targeting process is the integration of the transposable element including the polynucleotide of interest (e.g. encoding a protein or virus particle) via the transposase in transcriptionally active chromatin. This, in turn, allows the generation of high producer cell lines for the production of proteins (e.g. therapeutic proteins) or biopharmaceutical products based on virus particles.

The term “polynucleotide”, as used herein, means a polymer of deoxyribonucleotide bases or ribonucleotide bases and includes DNA and RNA molecules, both sense and anti-sense strands. In detail, the polynucleotide may be DNA, both cDNA and genomic DNA, RNA, mRNA, cRNA or a hybrid, where the polynucleotide sequence may contain combinations of deoxyribonucleotide or ribonucleotide bases, and combinations of bases including uracil, adenine, thymine, cytosine, guanine, inosine, xanthine, hypoxanthine, isocytosine and isoguanine. Polynucleotides may be obtained by chemical synthesis methods or by recombinant methods. Preferably, the polynucleotide is a DNA or mRNA molecule.

The terms “polypeptide” and “protein” are used interchangeably in the context of the present invention and refer to a long peptide-linked chain of amino acids.

The term “polypeptide fragment” as used in the context of the present invention refers to a polypeptide that has a deletion, e.g. an amino-terminal deletion, and/or a carboxy-terminal deletion, and/or an internally deletion compared to the full-length polypeptide.

The term “DNA binding/targeting domain”, as used herein, refers to a moiety that is capable of specifically binding to a DNA region (including chromosomal regions of higher order structure such as repetitive regions in the nucleus) and is, directly or indirectly, involved in mediating integration of a transposable element into said DNA region. The DNA region would preferably be defined by a nucleotide sequence which is unique within the respective genome.

The term “nuclear localization sequence/signal (NLS)”, as used herein, refers to a structure that tags a polypeptide for import into the cell nucleus by nuclear transport. Typically, this sequence/signal consists of one or more short sequences of positively charged lysines or arginines exposed on the surface of the polypeptide.

The term “polypeptide binding molecule”, as used herein, refers to a molecule that is capable of specifically binding to both, a transposase and a chromatin reader element, in particular chromatin reader domain. In a preferred embodiment of the present invention, the transposase is connected with the chromatin reader element, in particular chromatin reader domain, via a binding molecule to which the chromatin reader element, in particular chromatin reader domain, is attached. In this case, the polypeptide binding molecule functions as a bridging molecule.

The term “heterologous”, as used herein, refers to an element that is either derived from another natural source, e.g. another organism, or is taken out of its natural context, e.g. fused, attached, or coupled to another molecule, or is not normally found in nature. In particular, the term “heterologous polypeptide”, as used in the context of the present invention, refers to a polypeptide that is not normally found in nature. For example, the polypeptide comprising a transposase or a fragment or a derivative thereof having transposase function and at least one heterologous chromatin reader element is not found in nature, e.g. in a given cell. The term “heterologous nucleotide sequence”, as used in the context of the present invention, refers to a nucleotide sequence that is not normally found in nature, e.g. in a given cell. For example, the polynucleotide encoding the polypeptide comprising a transposase or a fragment or a derivative thereof having transposase function and at least one heterologous chromatin reader element is not found in nature, e.g. in a given cell. The term encompasses a nucleic acid wherein at least one of the following is true: (a) the nucleic acid that is exogenously introduced into a given cell (hence “exogenous sequence” even though the sequence can be foreign or native to the recipient cell), (b) the nucleic acid comprises a nucleotide sequence that is naturally found in a given cell (e.g. the nucleic acid comprises a nucleotide sequence that is endogenous to the cell) but the nucleic acid is either produced in an unnatural (e.g. greater than expected or greater than naturally found) amount in the cell, or the nucleotide sequence differs from the endogenous nucleotide sequence such that the same encoded protein (having the same or substantially the same amino acid sequence) as found endogenously is produced in an unnatural (e.g. greater than expected or greater than naturally found) amount in the cell, or (c) the nucleic acid comprises two or more nucleotide sequences or segments that are not found in the same relationship to each other in nature (e.g., the nucleic acid is recombinant).

The term “heterologous chromatin reader element, in particular chromatin reader domain”, as used herein in connection with a transposase or a fragment or a derivative thereof having transposase function, refers to an amino acid sequence that is normally not found intimately associated with a transposase, a fragment or a derivative thereof having transposase function in nature. A heterologous chromatin reader element may contain one or more than one protein domain within one or more polypeptide chains. A polypeptide comprising a transposase, a fragment or a derivative thereof having transposase function and a chromatin reader element, in particular chromatin reader domain, may also be designated as recombinant/artificial polypeptide.

The terms “heterologous DNA binding domain” or “heterologous nuclear localization sequence (NLS)” or “heterologous binding molecule”, as used herein in connection with a transposase or a fragment or a derivative thereof having transposase function, refer to amino acid sequences that are normally not found intimately associated with a transposase, or a fragment or a derivative thereof having transposase function in nature.

The term “linker”, as used herein, refers to a proteinaceous stretch of amino acids, e.g. of at least 2, 3, 4, or 5 amino acids, which does not fulfil a biological function within a host organism such as a cell. The function of a linker is to tether or combine two different polypeptides or domains or polypeptides and domains allowing these polypeptides or domains or polypeptides and domains to exert their biological functions that they would exert without being attached to said linker (such as binding to a chromatin target sequence, to DNA or to a different polypeptide or to excise and/or integrate polynucleotides).

The term “polynucleotide of interest”, as used herein, relates to a nucleotide sequence. The nucleotide sequence may be a RNA or DNA sequence, preferably the nucleotide sequence is a DNA sequence. In accordance with the method of the present invention, the polynucleotide of interest may encode for a product of interest. A product of interest may be a polypeptide of interest, e.g. a protein, or a RNA of interest, e.g. a mRNA or a functional RNA, e.g. a double stranded RNA, microRNA, or siRNA. Functional RNAs are frequently used to silence a corresponding target gene. Preferably, the polynucleotide of interest is operatively liked to suitable regulatory sequences (e.g. a promoter) which are well known and well described in the art and which may affect the transcription of the polynucleotide of interest.

The level of expression of a desired product in a host organism, e.g. host cell, may be determined on the basis of either the amount of corresponding mRNA that is present in the cell, or the amount of the desired product encoded by polynucleotide of interest. For example, mRNA transcribed from a selected sequence can be quantitated by PCR or by Northern hybridization. Polypeptides can be quantified by various methods, e.g. by assaying for the biological activity of the polypeptides (e.g. by enzyme assays), or by employing assays that are independent of such activity, such as western blotting, ELISA, or radioimmunoassay, using antibodies that recognize and bind to the protein. The polynucleotide of interest is preferably selected from the group consisting of a polynucleotide encoding a polypeptide, a non-coding polynucleotide, a polynucleotide comprising a promoter sequence, a polynucleotide encoding a mRNA, a polynucleotide encoding a tag, and a viral polynucleotide. The polynucleotide of interest is preferably a heterologous/exogenous polynucleotide.

The term “expression control sequences”, as used herein, refers to nucleotide sequences which affect the expression of coding sequences to which they are operably linked in a host organism, e.g. host cells. Expression control sequences are sequences which control the transcription, e.g. promoters, TATA-box, enhancers, UCOE or MAR elements, polyadenylation signals, post-transcriptionally active elements, e.g. RNA stabilising elements, RNA transport elements and translation enhancers.

The term “operably linked”, as used herein, means that one nucleotide sequence is linked to a second nucleotide sequence in such a way that in-frame expression of a corresponding fusion or hybrid protein can be affected avoiding frame-shifts or stop codons. This term also means the linking of expression control sequences to a coding nucleotide sequence of interest (e.g. coding for a protein) to effectively control the expression of said sequence. This term further means the linking of a nucleotide sequence encoding an affinity tag or marker tag to a coding nucleotide sequence of interest (e.g. coding for a protein).

The term “host cell”, as used herein, refers to any cell which may be used for protein and/or virus production. It also refers to any cell which may be the host for the polypeptide, polynucleotide and/or transposable element described herein. The cell may be a prokaryotic or an eukaryotic cell. Preferably, the cell is an eukaryotic cell. More preferably, the eukaryotic cell is a vertebrate, a yeast, a fungus, or an insect cell. The vertebrate cell may be a mammalian, a fish, an amphibian, a reptilian cell or an avian cell. The avian cell may be a chicken, a quail, a goose, or a duck cell such as a duck retina cell or duck somite cell. Even more preferably, the vertebrate cell is a mammalian cell. Most preferably, the mammalian cell is selected from the group consisting of a Chinese hamster ovary (CHO) cell (e.g. CHO-K1/CHO-S/CHO-DUXB11/CHO-DG44 cell), a human embryonic kidney (HEK293) cell, a HeLa cell, a A549 cell, a MRC5 cell, a WI38 cell, a BHK cell, and a Vero cell. The cell may also be comprised in/part of an organism. Said organism may be a prokaryotic or an eukaryotic organism. Preferably, the organism is an eukaryotic organism. More preferably, said organism may be a fungus, an insect, or a vertebrate. The vertebrate may be a bird (e.g. a chicken, quail, goose, or duck), a canine, a mustela, a rodent (e.g. a mouse, rat or hamster), an ovine, a caprine, a pig, a bat (e.g. a megabat or microbat) or a human/non-human primate (e.g. a monkey or a great ape). Most preferably the organism is a mammal such as a mouse, a rat, a pig, or a human/non-human primate.

### EMBODIMENTS OF THE INVENTION

The present inventors surprisingly found that an artificial transposable element comprising at least one polynucleotide of interest can effectively be targeted to active chromatin via a transposase coupled with at least one heterologous chromatin reader element. The present inventors surprisingly established, for the first time, a targeting system comprising an artificial transposable element comprising at least one polynucleotide of interest and a polypeptide comprising a transposase coupled with at least one heterologous chromatin reader element for the production of proteins and viruses in high yields. The present inventors found that the higher protein levels were not the result of higher transgene copy number but the result of efficient transgene integration into highly active genomic loci.

Thus, in a first aspect, the present invention relates to a polypeptide comprising a transposase or a fragment or a derivative thereof having transposase function and at least one chromatin reader element (CRE) (e.g. at least 1 or 2 CRE(s)). Said polypeptide is able to enhance insertion site selection in chromatin structures. It is preferred that the at least one chromatin reader element (CRE) is a heterologous chromatin reader element (CRE). It is, alternatively or additionally, preferred that the polypeptide is a recombinant polypeptide.

The polypeptide may be a molecule comprising a transposase and at least one heterologous CRE which can either be translated as a single chain polypeptide from the same nucleic acid molecule, e.g. mRNA molecule, or can be produced by separate translation of the transposase and the at least one heterologous CRE and subsequent coupling, e.g. by adhesion forces or chemically. In the first case, the at least one CRE is fused/attached to the transposase. In the second case, the at least one CRE is linked/coupled to the transposase. The preferred linkage is a covalent linkage. The polypeptide may be designated as recombinant/artificial polypeptide. Preferably, the polypeptide is a single chain polypeptide which may also be designated as hybrid polypeptide or fusion polypeptide.

In one embodiment, the at least one heterologous CRE is connected to the transposase. Preferably, the at least one heterologous CRE is connected to the transposase via a linker. The connection may be a linkage/coupling or a fusion/attachment. In particular, when the linker is present, the at least one CRE is linked/coupled or fused/attached to the transposase via the linker. If the polypeptide is produced as a single chain polypeptide (which may also be designated as a hybrid polypeptide or fusion polypeptide), the CRE is attached/fused to the transposase via the linker. If the polypeptide is produced by separate translation of the CRE and the transposase and subsequent coupling, e.g. by adhesion forces or chemically, the CRE is linked/coupled to the transposase via the linker. The preferred linkage is a covalent linkage.

In one preferred embodiment, the at least one heterologous CRE is connected to the N-terminus of the transposase, to the C-terminus of the transposase, or to the N-terminus and C-terminus of the transposase. Preferably, the at least one heterologous CRE is connected to the N-terminus of the transposase, to the C-terminus of the transposase, or to the N-terminus and C-terminus of the transposase via a linker.

In one preferred embodiment, the at least one heterologous CRE forms the N-terminus of the polypeptide, the C-terminus of the polypeptide, or the N-terminus and C-terminus of the polypeptide and is particularly coupled to the transposase via a linker.

The heterologous CREs forming the N-terminus of the transposase/polypeptide and the C-terminus of the transposase/polypeptide may be identical or different. They may be coupled to the transposase/polypeptide via identical or different linkers.

As mentioned above, one or more linkers may be comprised in the polypeptide to connect the one or more chromatin reader elements with the transposase. For example, one linker may be comprised to connect the N-terminus of the transposase with the CRE, one linker may be comprised to connect the C-terminus of the transposase with the CRE, or one linker may be comprised to connect the N-terminus of the transposase with a CRE and one another (identical or different) linker may be comprised to connect the C-terminus of the transposase with another (identical or different) CRE. Said linker may comprise at least 2, 3, 4, or 5 amino acids. Preferably, the linker is a flexible linker. More preferably, the linker is a glycine linker, a serine-glycine linker, a linker having an amino acid sequence according to SEQ ID NO: 22 or an amino acid sequence having at least 90%, e.g. at least 90, 91, 92, 93, 94, 95, 96, 97, 98, or 99%, sequence identity thereto, or a linker having an amino acid sequence according to SEQ ID NO: 23 or an amino acid sequence having at least 90%, e.g. at least 90, 91, 92, 93, 94, 95, 96, 97, 98, or 99%, sequence identity thereto.

In one alternatively preferred embodiment, the CRE is coupled/connected to the transposase via a binding molecule/moiety (instead of a linker). The molecule/moiety binding the CRE is preferably connected to the N-terminus or C-terminus of the transposase. Said binding molecule/moiety interacts with the transposase as well as with the CRE.

In one preferred embodiment, the at least one heterologous CRE is a chromatin reader domain (CRD). Preferably, the at least one heterologous CRD is a naturally occurring CRD. The (naturally occurring) chromatin reader domain may be a bromodomain, a chromodomain, a plant homeodomain (PHD) zinc finger, a WD40 domain, a tudor domain, double/tandem tudor domain, a MBT domain, an ankyrin repeat domain, a zf-CW domain, or a PWWP domain. More preferably, the (naturally occurring) CRD recognises histone methylation degree (e.g. mono-methylation, di-methylation, or tri-methylation of amino acids such as lysine or arginine) and/or acetylation state of histones. Even more preferably, the (naturally occurring) CRD recognising histone methylation degree is a plant homeodomain (PHD) type zinc finger, or the (naturally occurring) CRD recognising the acetylation state of histones is a bromodomain. Most preferably, the PHD type zinc finger is a transcription initiation factor TFIID subunit 3 PHD, e.g. having an amino acid sequence according to SEQ ID NO: 20 or an amino acid sequence having at least 90%, e.g. 90, 91, 92, 93, 94, 95, 96, 97, 98, or 99%, sequence identify thereto, or the bromodomain is a histone acetyltransferase domain, like a histone acetyltransferase KAT2A domain, e.g. having an amino acid sequence according to SEQ ID NO: 21 or an amino acid sequence having at least 90%, e.g. 90, 91, 92, 93, 94, 95, 96, 97, 98, or 99%, sequence identify thereto. The domain variants are functionally active domain variants, i.e. they are still able to function a chromatin reader domains. An alternative (naturally occurring) chromatin reader domain that recognizes histone methylation degree may be, for example, a chromodomain, aWD40 domain, a tudor domain, a double/tandem tudor domain, a MBT domain, an ankyrin repeat domain, a zf-CW domain, or a PWWP domain.

For example,
a RHD or bromodomain forms/is comprised at the N-terminus of the transposase and is particularly coupled to the transposase via a linker,
a RHD or bromodomain forms/is comprised at the C-terminus of the transposase and is particularly coupled to the transposase via a linker,
a RHD forms/is comprised at the N-terminus and a RHD forms/is comprised at the C-terminus of the transposase, both are particularly coupled to the transposase via a linker,
a bromodomain forms/is comprised at the N-terminus and a bromodomain forms/is comprised at the C-terminus of the transposase, both are particularly coupled to the transposase via a linker,
a RHD forms/is comprised at the N-terminus and a bromodomain forms/is comprised at the C-terminus of the transposase, both are particularly coupled to the transposase via a linker, or
a bromodomain forms/is comprised at the N-terminus and a RHD forms/is comprised at the C-terminus of the transposase, both are particularly coupled to the transposase via a linker.
The nucleotide sequences and the corresponding amino acid sequences of preferred polypeptides comprising a transposase and at least one heterologous chromatin reader domain are listed under SEQ ID NO: 1 and SEQ ID NO: 2 for Taf3-haPB, SEQ ID NO: 3 and SEQ ID NO: 4 for KATA2A-PBw-TAF3, under SEQ ID NO: 5 and SEQ ID NO: 6 for PBw, under SEQ ID NO: 7 and SEQ ID NO: 8 for TAF3-PBw, under SEQ ID NO: 9 and SEQ ID NO: 10 for PBw-TAF3, under SEQ ID NO: 11 and SEQ ID NO: 12 for KAT2A-PBw, under SEQ ID NO: 13 and SEQ ID NO: 14 for haPB, under SEQ ID NO: 15 and SEQ ID NO: 16 for KATA2A-haPB-TAF3, under SEQ ID NO: 29 and SEQ ID NO: 30 for KATA2A-haPB, and under SEQ ID NO: 31 and SEQ ID NO: 32 for haPB-TAF3. Variants (on the nucleotide sequence as well as amino acid level) of the above-mentioned sequences are also encompassed. Said variants have at least 90%, e.g. 90, 91, 92, 93, 94, 95, 96, 97, 98, or 99%, sequence identify to the above-mentioned sequences. The variants are functionally active variants or code for functionally active variants. Functionally active variants are still able to detect and bind transcriptionally active chromatin (euchromatin) and are still able to excise and insert transposable elements.

In one alternatively preferred embodiment, the chromatin reader element is an artificial chromatin reader element (CRE). Preferably, the artificial CRE recognises histone tails with specific methylated and/or acetylated sites. More preferably, the artificial CRE is selected from the group consisting of a micro antibody, a single chain antibody, an antibody fragment, an affibody, an affilin, an anticalin, an atrimer, a DARPin, a FN2 scaffold, a fynomer, and a Kunitz domain.

The transposase may be a transposase of class I (retrotransposase) or a transposase of class II (DNA transposase). In case of a transposase of class I, the transposase may also be designated as integrase. In one preferred embodiment, the transposase is a class II transposase (DNA transposase). In one more preferred embodiment, the transposase is a PiggyBac transposase, a sleeping beauty transposase, or a Tol2 transposase. Preferably, the PiggyBac transposase is a wild-type PiggyBac transposase, a hyperactive PiggyBac transposase, a wild-type PiggyBac-like transposase, or a hyperactive PiggyBac-like transposase. The wild-type PiggyBac transposase has more preferably an amino acid sequence according to SEQ ID NO: 6 or an amino acid sequence having at least 90%, e.g. at least 90, 91, 92, 93, 94, 95, 96, 97, 98, or 99%, sequence identity thereto. The wild-type PiggyBac transposase variants are functionally active variants, i.e. they are still able to function as transposases (excision as well as integration of polynucleotides). The PiggyBac-like transposase is more preferably selected from the group consisting of PiggyBat, PiggyBac-like transposase from Xenopus tropicalis, and PiggyBac-like transposase from Bombyx mori.

In one further preferred embodiment, the polypeptide further comprises at least one heterologous DNA binding domain (e.g. at least 1 or 2 DNA binding domain(s)).

In one also preferred embodiment, the polypeptide further comprises a heterologous nuclear localization signal (NLS). The NLS may form the N-terminus or the C-terminus of the transposase/polypeptide.

The polypeptide described above is preferably a heterologous polypeptide.

In a second aspect, the present invention relates to a polynucleotide encoding the polypeptide according to the first aspect. Said polynucleotide is preferably DNA or RNA such as mRNA.

In a third aspect, the present invention relates to a vector comprising the polynucleotide according to the second aspect. The terms “vector” and “plasmid” can interchangeable be used herein. The vector may be a viral or non-viral vector. Preferably, the vector is an expression vector. The expression of the polynucleotide encoding the polypeptide according to the first aspect is preferably controlled by expression control sequences. Expression control sequences may be sequences which control the transcription, e.g. promoters, enhancers, UCOE or MAR elements, polyadenylation signals, post-transcriptionally active elements, e.g. RNA stabilising elements, RNA transport elements and translation enhancers. Said expression control sequences are known to the skilled person. For example, as promoters, CMV or PGK promoters may be used.

In a fourth aspect, the present invention relates to a method for producing a cell, in particular transgenic cell, comprising the steps of:
(i) providing a cell, and
(ii) introducing
   a transposable element comprising at least one polynucleotide of interest, and
   a polypeptide according to the first aspect,
   a polynucleotide according to the second aspect, or
   a vector according to the third aspect
   into the cell, thereby producing/obtaining the cell, in particular transgenic cell.

The method may be an in vitro or in vivo method. Preferably, the method is an in vitro method.

Naturally, a transposable element includes a polynucleotide encoding a functional transposase that catalyses excision and insertion. The transposable element referred to in step (ii) of the above-mentioned method is, however, devoid of a polynucleotide encoding a functional transposase. The transposable element does not comprise the complete sequence encoding a functional, preferably a naturally occurring, transposase. Preferably, the complete sequence encoding a functional, preferably a naturally occurring, transposase or a portion thereof, is deleted from the transposable element. Instead of a polynucleotide encoding a functional transposase, at least one polynucleotide of interest, e.g. at least one exogenous/heterologous polynucleotide, is part of the transposable element described above. Thus, said transposable element may also be designated as recombinant/artificial transposable element.

The transposase or a fragment or a derivative thereof having transposase function connected to at least one heterologous chromatin reader element (CRE) is provided in step (ii) of the above-mentioned method in trans, e.g. as a polypeptide according to the first aspect, as a polynucleotide according to the second aspect, or comprised in a vector according to the third aspect.

The introduction of the transposable element comprising at least one polynucleotide of interest may take place via electroporation, transfection, injection, lipofection, or (viral) infection. The transposable element comprising at least one polynucleotide of interest may be introduced transiently or stably into the cell. In the first case, the transposable element comprising at least one polynucleotide of interest is introduced as extrachromosomal element, e.g. as linear DNA molecule, plasmid DNA, episomal DNA, viral DNA, or viral RNA. In the second case, the transposable element comprising at least one polynucleotide of interest is stably introduced/inserted into the genome of the cell. Preferably, the transposable element comprising at least one polynucleotide of interest is transiently introduced into the cell. More preferably, the transposable element comprising at least one polynucleotide of interest is comprised in a vector. The person skilled in the art is well informed about molecular biological techniques, such as microinjection, electroporation or lipofection, for introducing the transposable element into a cell and knows how to perform these techniques.

The introduction of the polypeptide according to the first aspect, the polynucleotide according to the second aspect, or the vector according to the third aspect may also take place via electroporation, transfection, injection, lipofection, and/or (viral) infection.

If a polynucleotide is introduced into the cell, the polynucleotide is subsequently transcribed and translated into the polypeptide in the cell. If a vector comprising the polynucleotide is introduced into the cell, the polynucleotide is subsequently transcribed from the vector and translated into the polypeptide in the cell. The polynucleotide may be DNA or RNA such as mRNA. Also viral DNA or RNA may be introduced. The polynucleotide may be introduced transiently or stably into the cell. In the first case, the polynucleotide is introduced as extrachromosomal polynucleotide, e.g. as linear DNA molecule, circular DNA molecule, plasmid DNA, viral DNA, in vitro synthesised/transcribed RNA, or viral RNA. In the second case, the polynucleotide is stably introduced/inserted into the genome of the cell. Preferably, the polynucleotide is transiently introduced into the cell. More preferably, the polynucleotide is comprised in a vector, in particular in an expression vector. The viral DNA or RNA sequences may also be introduced as part of a vector or in form of a vector. It is particularly preferred that the polynucleotide is operably linked to a heterologous promoter allowing the transcription of the transposase, or a fragment or a derivative thereof having transposase function and the at least one chromatin reader element within the cell or from a vector, e.g. expression vector or a vector used for in vitro transcription, comprised in the cell.

The person skilled in the art is well informed about molecular biological techniques, such as microinjection, electroporation or lipofection, for introducing polypeptides or nucleic acid sequences encoding polypeptides into a cell and knows how to perform these techniques.

In one preferred embodiment, the transposable element comprising at least one polynucleotide of interest is comprised in/part of a polynucleotide molecule, preferably a vector. In this case, the polynucleotide according to the second aspect is also preferably comprised in/part of a (different) polynucleotide molecule, preferably a (different) vector. Thus, it is preferred that the polynucleotide according to the second aspect and the transposable element are on separate polynucleotide molecules, preferably vectors. This allows the adaptation of transposase and transposable element plasmid amounts to achieve a few or as many integrations peer cell as desired.

In one alternatively preferred embodiment, the transposable element comprising at least one polynucleotide of interest and the polynucleotide according to the second aspect are comprised in/part of a (the same) polynucleotide molecule, preferably a vector. In this case, it is preferred that the polynucleotide according to the second aspect is located external to the region of the at least one polynucleotide of interest. Preferably, said polynucleotide is operably linked to a heterologous promoter allowing the transcription of the transposase, or a fragment or a derivative thereof having transposase function and the at least one chromatin reader element from the polynucleotide molecule, preferably vector.

The transposable element referred to in step (ii) of the above-mentioned method retains sequences that are required for mobilization by the transposase provided in trans. These are the repetitive sequences at each end of the transposable element containing the binding sites for the transposase allowing the excision from the genome. Thus, in one embodiment, the transposable element comprises terminal repeats (TRs). In one further embodiment, the at least one polynucleotide of interest is flanked by TRs. For example, the transposable element referred to in step (ii) of the above mentioned method comprises a first transposable element-specific terminal repeat and a second transposable element-specific terminal repeat downstream of the first transposable element-specific terminal repeat. The at least one polynucleotide of interest is located between the first transposable element-specific terminal repeat and the second transposable element-specific terminal repeat. Preferably, the terminal repeats are inverted terminal repeats (ITRs) or long terminal repeats (LTRs). In this respect, it should be noted that the transposase provided in trans is specific for the transposable element. In other words, the transposable element is specifically recognized by the transposase. A transposase of class II (DNA transposase), for example, recognises a TA dinucleotide at each end of the transposable element, particularly within the repetitive sequences/terminal repeats of the transposable element. It also recognises a TA dinucleotide in the target sequence.

As mentioned above, the transposable element comprising at least one polynucleotide of interest and the polynucleotide according to the second aspect are comprised in/part of a (the same) polynucleotide molecule, preferably a vector. In this case, it is preferred that the polynucleotide according to the second aspect is located external to the region of the at least one polynucleotide of interest. It is particularly preferred that the polynucleotide according to the second aspect is located outside of the terminal repeats, e.g. inverted terminal repeats (ITRs) or long terminal repeats (LTR), flanking the at least one polynucleotide of interest.

The transposable element may be derived from a prokaryotic or an eukaryotic transposable element, wherein the latter is preferred.

The transposable element may be a Class II or a DNA/DNA-based transposable element. The DNA/DNA-based transposable element comprises inverted terminal repeats (ITRs). It is recognized by a transposase of class II (DNA transposase). The transposable element may also be a Class I or a retrotransposable element. The retrotransposable element may be a long terminal repeat (LTR) retrotransposable element. The LTR retrotransposable element comprises long terminal repeats (LTRs). It is recognized by a transposase of class I (retrotransposase). Said transposase may also be designated as integrase.
As mentioned above, class II or DNA-based transposable elements contain inverted terminal repeats (ITRs) at either end. Conservative DNA-based transposable elements move by a cut-and-paste mechanism. This requires a transposase, inverted repeats at the ends of the transposable element and a target sequence on the new host DNA molecule. The transposase is provided in the above mentioned method in trans. It catalysis the excision of the transposable element from the current location and the integration of the excised transposable element into the genome of a cell. In the cut-and-paste mechanism, the transposase specifically binds to the inverted terminal repeats of the transposable element and cuts the transposable element out of the current location, e.g. vector. The transposase then locates the transposable element, cuts the target DNA backbone and then inserts the transposable element. Usually, two transposase monomers are involved in the excision of the transposable element, one transposase monomer at each end of the transposable element. Finally, the transposase dimer in complex with the excised transposable element reintegrates the transposable element in the DNA of a cell.

In one preferred embodiment, the transposable element is a class II or DNA-based transposable element. In one more preferred embodiment, the transposable element is a PiggyBac transposable element, a sleeping beauty transposable element, or a Tol2 transposable element. Preferably, the PiggyBac transposable element is a wild-type PiggyBac transposable element, a hyperactive PiggyBac transposable element, a wild-type PiggyBac-like transposable element, or a hyperactive PiggyBac-like transposable element. The PiggyBac-like transposable element is more preferably selected from the group consisting of a PiggyBat transposable element, a PiggyBac-like transposable element from Xenopus tropicalis, and a PiggyBac-like transposable element from Bombyx mori. The PiggyBac DNA transposable element is, for example, used technologically and commercially in genetic engineering by virtue of its property to efficiently transpose between vectors and chromosomes.

In one further preferred embodiment, the transposon-specific inverted terminal repeats comprise the PiggyBac minimal ITR. In one more preferred embodiment, the first transposon-specific inverted terminal repeat comprises the sequence according to SEQ ID NO: 24 or a sequence having at least 90%, e.g. at least 90, 91, 92, 93, 94, 95, 96, 97, 98, or 99%, sequence identity thereto, and/or the second transposon-specific inverted terminal repeat comprises the sequence according to SEQ ID NO: 25 or a sequence having at least 90%, e.g. at least 90, 91, 92, 93, 94, 95, 96, 97, 98, or 99%, sequence identity thereto. The PiggyBac minimal ITR variants are functionally active variants, i.e. they can still be recognised by a transposase specific for the PiggyBac minimal ITR.

The cell may be a prokaryotic or an eukaryotic cell. Preferably, the cell is an eukaryotic cell. More preferably, the eukaryotic cell is a vertebrate, a yeast, a fungus, or an insect cell. The vertebrate cell may be a mammalian, a fish, an amphibian, a reptilian cell or an avian cell. The avian cell may be a chicken, quail, goose, or duck cell such as a duck retina cell or duck somite cell. Even more preferably, the vertebrate cell is a mammalian cell. Most preferably, the mammalian cell is selected from the group consisting of a Chinese hamster ovary (CHO) cell (e.g. CHO-K1/CHO-S/CHO-DUXB11/CHO-DG44 cell), a human embryonic kidney (HEK293) cell, a HeLa cell, a A549 cell, a MRC5 cell, a WI38 cell, a BHK cell, and a Vero cell.

The cell may be an isolated cell (such as in a cell culture or in a cell line, e.g. stable cell line). The cell may also be a cell of a tissue outside of an organism. The transgenic cell may, however, subsequently be inserted into an organism. Insertion of the transgenic cell into the organisms may be effected by infusion or injection or further means well known to the person skilled in the art.

The cell may also be part of/comprised in an organism, e.g. eukaryotic multicellular organism. In this case, the insertion of a transposable element comprising at least one polynucleotide of interest, and a polypeptide according to the first aspect, a polynucleotide according to the second aspect, or a vector according to the third aspect is effected in vivo. In vivo polypeptide/polynucleotide/transposable element delivery can be accomplished by injection (either locally or systemically). The polynucleotide/transposable element can be, for example, in the form of naked DNA, DNA complexed with liposomes, PEI or other condensing agents, or can be incorporated into infectious particles (viruses or virus-like particles). Polynucleotide/transposable element delivery can also be done using electroporation or with gene guns or with aerosols.

Said organism may be a prokaryotic or an eukaryotic organism. Preferably, said organism is an eukaryotic organism. More preferably, said organism may be a fungus, an insect, or a vertebrate. The vertebrate may be a bird (e.g. a chicken, quail, goose, or duck), a canine, a mustela, a rodent (e.g. a mouse, rat or hamster), an ovine, a caprine, a pig, a bat (e.g. a megabat or microbat) or a human/non-human primate (e.g. a monkey or a great ape). Most preferably the organism is a mammal such as a mouse, a rat, a pig, or a human/non-human primate.

In one embodiment, the at least one polynucleotide of interest is selected from the group consisting of a polynucleotide encoding a polypeptide, a non-coding polynucleotide, a polynucleotide comprising a promoter sequence, a polynucleotide encoding a mRNA, a polynucleotide encoding a tag, and a viral polynucleotide.

The polypeptide encoded by the polynucleotide may be a therapeutically active polypeptide, e.g. an antibody, an antibody fragment, a monoclonal antibody, a virus protein, a virus protein fragment, an antigen, a hormone. The polypeptide may further be used for gene therapy, e.g. of monogenic diseases. In this case, the polynucleotide encoding the polypeptide is operably linked with a tissue-specific promoter. The polypeptide may also be used for cell therapy, in particularly ex vivo. The cells may be pluripotent stem cells (iPSC), human embryonic stem (hES) cells, human hematopoietic stem cells (HSCs), or human T lymphocytes.
The non-coding polynucleotide may be useful in the targeted disruption of a gene.
The polynucleotide comprising promoter sequences may allow the activation of gene expression if the transposon inserts close to an endogenous gene.
The polynucleotide may be transcribed into mRNA or a functional noncoding RNA e.g. a miRNAi or gRNA.
The polynucleotide may comprise a sequence tag to identify the insertion site of the transposable element.
The viral polynucleotide may be used for the production of biopharmaceutical products based on virus particles.

The transposable element and/or the vector comprising the transposable element may further comprise elements that enhance expression (e.g. nuclear export signals, promoters, introns, terminators, enhancers, elements that affect chromatin structure, RNA export elements, IRES elements, CHYSEL elements, and/or Kozak sequences), selectable marker (e.g. DHFR, puromycine, hygromycin, zeocin, blasticidin, and/or neomycin), markers for in vivo monitoring (e.g. GFP or beta-galactosidase), a restriction endonuclease recognition site (e.g. a site for insertion of an exogenous nucleotide sequence such as a multiple cloning site), a recombinase recognition site (e.g. LoxP (recognized by Cre), FRT (recognized by Flp), or AttB/AttP (recognized by PhiC31)), insulators (e.g. MARs or UCOEs), viral replication sequences (e.g. SV40 ori), and/or a sequence compatible to a DNA binding domain, in particular for targeting via an additional binding molecule with chromatin reader domain and DNA binding domain properties (“bridging”).

In the above-described method, not only one but also more than one transposable element may be inserted into the cell. The transposable elements may differ from each other, e.g. as they comprise different polynucleotides of interest. This is specifically desired in cases were two ORFs encoding antibody heavy chains (HC) or antibody light chains (LC) have to be introduced into the cell. In this case, the two or more ORFs are comprised in the same or on separate transposable elements, preferably on separate transposable elements.

In the fifth aspect, the present invention relates to a cell, in particular transgenic cell, obtainable/producible by the method of the fourth aspect.

In a sixth aspect, the present invention relates to the use of a cell, in particular transgenic cell, of the fifth aspect for the production of a protein or virus. The proteins may be therapeutic proteins. The virus may be a vector (viral vector).

In a seventh aspect, the prevent invention relates to a kit comprising
(i) a transposable element comprising a cloning site for inserting at least one polynucleotide of interest, and
(ii) a polypeptide according to the first aspect,
   a polynucleotide according to the second aspect,
   a vector according to the third aspect, or
   at least one heterologous CRE and a polypeptide comprising a transposase or a fragment or a derivative thereof having transposase function.

The transposable element provided with the kit/comprised in the kit is devoid of a polynucleotide encoding a functional transposase. The transposable element does not comprise the complete sequence encoding a functional, preferably a naturally occurring, transposase. Preferably, the complete sequence encoding a functional, preferably a naturally occurring, transposase or a portion thereof, is deleted from the transposable element. Instead of a polynucleotide encoding a functional transposase, the transposable element comprises a cloning site (in particular at least one cloning site) for inserting at least one polynucleotide of interest. The type of the polynucleotide of interest which is finally introduced into the transposable element depends on the end user. The transposable element may be a recombinant, an artificial, and/or a heterologous transposable element.

The transposase is an independent or a distinct component of the kit. It is provided with the kit/comprised in the kit connected to a heterologous chromatin reader element (CRE) as a polypeptide according to the first aspect, as a polynucleotide according to the second aspect, or comprised in a vector according to the third aspect (see item (ii)).

In an alternative, a polypeptide comprising a transposase or a fragment, or a derivative thereof having transposase function is provided with the kit/comprised in the kit without being connected to a chromatin reader element (CRE), in particular chromatin reader domain (CRD). In this specific case, the polypeptide comprising a transposase or a fragment, or a derivative thereof having transposase function and the chromatin reader element (CRE), in particular chromatin reader domain (CRD), is provided with the kit/comprised in the kit as independent or distinct components. Preferably, the CRE, in particular CRD, is associated with a binding molecule/moiety which is—after introduction into a cell—able to bind the transposase (e.g. via the N-terminus or C-terminus) forming a transposase, binding molecule/moiety and CRE, in particular CRE, complex. This, of course, requires that the polypeptide comprising a transposase, or a fragment, or a derivative thereof having transposase function comprises a binding domain allowing the binding molecule/moiety associated with the CRE, in particular CRD, to bind. This binding domain is preferably a protein binding domain. Alternatively, the CRE, in particular CRD, is associated with a binding molecule/moiety which is—after introduction into a cell—able to bind the transposable element. This, of course, requires that the transposable element comprises a binding domain allowing the binding molecule/moiety associated with the CRE, in particular CRD, to bind. This binding domain is preferably a DNA binding domain. The polypeptide comprising a transposase or a fragment or a derivative thereof having transposase function may be a recombinant, an artificial, and/or a heterologous polypeptide.

The transposable element may be provided with the kit/comprised in the kit as a linear DNA molecule, plasmid DNA, episomal DNA, viral DNA, or viral RNA. It is preferred that the transposable element comprises a heterologous promoter which allows, after integration of the at least one polynucleotide of interest into the cloning site, the transcription of the at least one polynucleotide of interest. Preferably, the transposable element is comprised in a vector.

The polynucleotide according to the second aspect may also be provided with the kit/comprised in the kit as a linear DNA molecule, a circular DNA molecule, plasmid DNA, viral DNA, in vitro synthesised/transcribed RNA or viral RNA. It is preferred that the polynucleotide is operably linked to a heterologous promoter allowing the transcription of the transposase, or a fragment or a derivative thereof having transposase function and the at least one chromatin reader element. Preferably, the polynucleotide is comprised in a vector, in particular an expression vector or a vector for in vitro transcription.

The transposable element and the polynucleotide according to the second aspect may be part of different vectors. This allows the adaptation of transposase and transposable element plasmid amounts to achieve a few or as many integrations peer cell as desired.

The transposable element and the polynucleotide according to the second aspect may also be part of the same vector. In this case, it is preferred that the polynucleotide is located external to the cloning site for inserting at least one polynucleotide of interest.

The transposable element provided with the kit/comprised in the kit retains sequences that are required for mobilization by the transposase provided in trans. These are the repetitive sequences at each end of the transposable element containing the binding sites for the transposase allowing the excision from the genome. Thus, in one embodiment, the transposable element comprises terminal repeats (TRs). In one further embodiment, the at least one polynucleotide of interest is flanked by TRs. For example, the transposable element referred to in step (ii) of the above mentioned method comprises a first transposable element-specific terminal repeat and a second transposable element-specific terminal repeat downstream of the first transposable element-specific terminal repeat. The cloning site for inserting at least one polynucleotide of interest is located between the first transposable element-specific terminal repeat and the second transposable element-specific terminal repeat. Preferably, the terminal repeats are inverted terminal repeats (ITRs) or long terminal repeats (LTRs). In this respect, it should be noted that the transposase provided with the kit/comprised in the kit is specific for the transposable element. In other words, the transposable element can specifically be recognized by the transposase. A transposase of class II (DNA transposase), for example, recognises a TA dinucleotide at each end of the transposable element, particularly within the repetitive sequences/terminal repeats of the transposable element. It also recognises a TA dinucleotide in the target sequence.

As mentioned above, the transposable element and the polynucleotide according to the second aspect may be part of the same vector. In this case, it is preferred that the polynucleotide is located external to the cloning site for inserting at least one polynucleotide of interest. It is particularly preferred that the polynucleotide according to the second aspect is located outside of the terminal repeats, e.g. inverted terminal repeats (ITRs) or long terminal repeats (LTR), flanking the cloning site for inserting the at least one polynucleotide of interest.

The transposable element provided with the kit/comprised in the kit may be derived from a prokaryotic or an eukaryotic transposable element, wherein the latter is preferred.

The transposable element may be a Class II or a DNA/DNA-based transposable element. The DNA/DNA-based transposable element comprises inverted terminal repeats (ITRs). It is recognized by a transposase of class II (DNA transposase). The transposable element may also be a Class I or a retrotransposable element. The retrotransposable element may be a long terminal repeat (LTR) retrotransposable element. The LTR retrotransposable element comprises long terminal repeats (LTRs). It is recognized by a transposase of class I (retrotransposase). Said transposase may also be designated as integrase.

In one preferred embodiment, the transposable element is a Class II or a DNA/DNA-based transposable element. In one more preferred embodiment, the transposable element is a PiggyBac transposable element, a sleeping beauty transposable element, or a Tol2 transposable element. Preferably, the PiggyBac transposable element is a wild-type PiggyBac transposable element, a hyperactive PiggyBac transposable element, a wild-type PiggyBac-like transposable element, or a hyperactive PiggyBac-like transposable element. The PiggyBac-like transposable element is more preferably selected from the group consisting of a PiggyBat transposable element, a PiggyBac-like transposable element from Xenopus tropicalis, and a PiggyBac-like transposable element from Bombyx mori.

The transposable element and/or the vector comprising the transposable element may further comprise elements that enhance expression (e.g. nuclear export signals, promoters, introns, terminators, enhancers, elements that affect chromatin structure, RNA export elements, IRES elements, CHYSEL elements, and/or Kozak sequences), selectable marker (e.g. DHFR, puromycine, hygromycin, zeocin, blasticidin, and/or neomycin), marker for in vivo monitoring (e.g. GFP or beta-galactosidase), a restriction endonuclease recognition site (e.g. a site for insertion of an exogenous nucleotide sequence such as a multiple cloning site), a recombinase recognition site (e.g. LoxP (recognized by Cre), FRT (recognized by Flp), or AttB/AttP (recognized by PhiC31)), insulators (e.g. MARs or UCOEs), viral replication sequences (e.g. SV40 ori), and/or a sequence compatible to a DNA binding domain, in particular for targeting via an additional binding molecule with chromatin reader domain and DNA binding domain properties (“bridging”).

The kit may comprise not only one but also more than one transposable element. The transposable elements may differ from each other, e.g. with respect to the cloning site and/or the specific composition of additional elements. This allows the cloning of diverse polynucleotides of interest into the different transposable elements.

In one embodiment, the kit is for the generation of a cell, in particular transgenic cell.

In one another embodiment, the kit further comprises instructions on how to generate the cell, in particular transgenic cell.

The kit may further comprise a container, wherein the single components of the kit are comprised. The kit may also comprise materials desirable from a commercial and user standpoint including a buffer(s), a reagent(s) and/or a diluent(s).

In an eight aspect, the present invention relates to a targeting system comprising
(i) a transposable element comprising at least one polynucleotide of interest, and a polypeptide according to the first aspect,
(ii) a transposable element comprising at least one polynucleotide of interest, and a polynucleotide according to the second aspect,
(iii) a transposable element comprising at least one polynucleotide of interest, and a vector according to the third aspect, or
(iv) a transposable element comprising at least one polynucleotide of interest,
   at least one heterologous chromatin reader element (CRE), optionally associated with the transposable element, and
   a polypeptide comprising a transposase or a fragment or a derivative thereof having transposase function.

The targeting system may be comprised in/part of a cell or may be introduced into a cell. The introduction of the targeting system into a cell may take place via electroporation, transfection, injection, lipofection, or (viral) infection.

The cell may be an isolated cell (such as in cell culture or in cell line, e.g. stable cell line). The cell may also be a cell of a tissue outside of an organism. The cell may further be part of/comprised in an organism, e.g. eukaryotic multicellular organism. In this case, the insertion of the targeting system is effected in vivo.

In an alternative, a polypeptide comprising a transposase or a fragment, or a derivative thereof having transposase function is comprised in the targeting system without being connected to a chromatin reader element (CRE), in particular chromatin reader domain (CRD) (see under (iv)). In this specific case, the polypeptide comprising a transposase or a fragment, or a derivative thereof having transposase function and the chromatin reader element (CRE), in particular chromatin reader domain (CRD), are comprised in the targeting system as distinct components. Preferably, the CRE, in particular CRD, is associated with a binding molecule/moiety which is—after introduction into a cell—able to bind the transposase (e.g. via the N-terminus or C-terminus) forming a transposase, binding molecule/moiety and CRE, in particular CRD, complex. This, of course, requires that the polypeptide comprising a transposase, or or a fragment, or a derivative thereof having transposase function comprises a binding domain allowing the binding molecule/moiety associated with the CRE, in particular CRD, to bind. This binding domain is preferably a protein binding domain. Alternatively, the CRE, in particular CRD, is associated with a binding molecule/moiety which is—after introduction into a cell—able to bind the transposable element. This, of course, requires that the transposable element comprises a binding domain allowing the binding molecule/moiety associated with the CRE, in particular CRD, to bind. This binding domain is preferably a DNA binding domain.

The polypeptide comprising a transposase or a fragment or a derivative thereof having transposase function may be a recombinant, an artificial, and/or a heterologous polypeptide.

In one embodiment, the transposable element comprising at least one polynucleotide of interest is comprised in/part of a polynucleotide molecule, preferably a vector.

In one alternative embodiment, the transposable element comprising at least one polynucleotide of interest and the polynucleotide according to the second aspect are comprised in/part of a polynucleotide molecule, preferably a vector.

The transposable element may be a recombinant, an artificial, and/or a heterologous transposable element.

In one preferred embodiment, the transposable element is a Class II or a DNA/DNA-based transposable element. In one more preferred embodiment, the transposable element is a PiggyBac transposable element, a sleeping beauty transposable element, or a Tol2 transposable element. Preferably, the PiggyBac transposable element is a wild-type PiggyBac transposable element, a hyperactive PiggyBac transposable element, a wild-type PiggyBac-like transposable element, or a hyperactive PiggyBac-like transposable element. The PiggyBac-like transposable element is more preferably selected from the group consisting of a PiggyBat transposable element, a PiggyBac-like transposable element from Xenopus tropicalis, and a PiggyBac-like transposable element from Bombyx mori.

Preferably, the chromatin reader element (CRE) is a chromatin reader domain (CRD).

As to further preferred embodiments of the transposable element, it is referred to the fourth or seventh aspect of the present invention.

In a further aspect, the present invention relates to a targeting system comprising (i) a transposable element comprising at least one polynucleotide of interest and (ii) a polypeptide comprising a transposase or a fragment or a derivative thereof having transposase function, characterized in that the transposable element and/or the polypeptide comprising a transposase or a fragment or a derivative thereof having transposase function is directly associated (preferably via covalent fusion/attachment) or indirectly associated (preferably via a binding molecule) with a heterologous chromatin reader element (CRE), preferably chromatin reader domain (CRD).

As to preferred embodiments of the transposable element, it is referred to the fourth and/or seventh aspect of the present invention.

In a further aspect, the present invention relates to a (transgenic) cell comprising

a transposable element comprising at least one polynucleotide of interest, and
a polypeptide according to the first aspect,
a polynucleotide according to the second aspect, or
a vector according to the third aspect.
As to further preferred embodiments with respect to the cell and the transposable element, it is referred to the fourth aspect of the present invention.

In a further aspect, the present invention relates to a (transgenic) cell comprising a heterologous transposable element which comprises at least one polynucleotide of interest, wherein the heterologous transposable element is predominantly, preferably exclusively, integrated/located in transcriptionally active genomic structures (euchromatin). More preferably, the heterologous transposable element is predominantly, preferably exclusively, integrated/located in (a) transcriptionally active promoter region(s). Said cell had been treated with a targeting system according to the eight aspect.

As to further preferred embodiments with respect to the cell and the transposable element, it is referred to the fourth aspect of the present invention.

Various modifications and variations of the invention will be apparent to those skilled in the art without departing from the scope of invention. Although the invention has been described in connection with specific preferred embodiments, it should be understood that the invention as claimed should not be unduly limited to such specific embodiments. Indeed, various modifications of the described modes for carrying out the invention which are obvious to those skilled in the art in the relevant fields are intended to be covered by the present invention.

### BRIEF DESCRIPTION OF THE FIGURES

The following Figures and examples are merely illustrative of the present invention and should not be construed to limit the scope of the invention as indicated by the appended claims in any way.

FIG. 1: Synthesised transposase constructs. PiggyBac wt (PBw): wt PiggyBac transposase, Trichoplusia ni, GenBank accession number #AAA87375.2; hyperactive PiggyBac (haPB): transposase mutated in I30V, G165S, M282V, N538K compared to wt PiggyBac transposase according to GenBank accession number #AAA87375.2; TAF3 PHD: TaflID sub III PHD domain, Homo sapiens, GenBank accession number #NP_114129.1 855 . . . 929; KAT2A Bromodomain: histone acetyltransferase KAT2A Bromodomain, Homo sapiens, GenBank accession number NP_066564.2 741 . . . 837; L1: Peptidelinker, KLGGGAPAVGGGPKKLGGGAPAVGGGPK SEQ ID NO: 22; L2: Peptidelinker, AAAKLGGGAPAVGGGPKAADKGAA SEQ ID NO: 23. The coding sequence (CDS) of Taf3-haPB is shown under SEQ ID NO: 1 and the coding sequence (CDS) of KATA2A-PBw-TAF3 is shown under SEQ ID NO: 3. SEQ ID NO: 2 shows the amino acid sequence of Taf3-haPB and SEQ ID NO: 4 shows the amino acid sequence of KATA2A-PBw-TAF3.

FIG. 2: Tested variants of PiggyBac fusion proteins. PiggyBac wt (PBw): wt PiggyBac transposase, Trichoplusia ni, GenBank accession number #AAA87375.2; Hyperactive PiggyBac (haPB): transposase mutated in I30V, G165S, M282V, N538K compared to wt PiggyBac transposase; TAF3 PHD: TaflID sub III PHD domain, Homo sapiens, GenBank accession number #NP_114129.1 855 . . . 929; KAT2A Bromodomain: histone acetyltransferase KAT2A Bromodomain, Homo sapiens, GenBank accession number NP_066564.2 741 . . . 837; L1: Peptidelinker, KLGGGAPAVGGGPKKLGGGAPAVGGGPK SEQ ID NO: 22; L2: Peptidelinker, AAAKLGGGAPAVGGGPKAADKGAA SEQ ID NO: 23. The nucleotide sequences and the corresponding amino acid sequences are listed under SEQ ID NO: 3 and SEQ ID NO: 4 for KATA2A-PBw-TAF3, under SEQ ID NO: 5 and SEQ ID NO: 6 for PBw, under SEQ ID NO: 7 and SEQ ID NO: 8 for TAF3-PBw, under SEQ ID NO: 9 and SEQ ID NO: 10 for PBw-TAF3, under SEQ ID NO: 11 and SEQ ID NO: 12 for KAT2A-PBw, under SEQ ID NO: 13 and SEQ ID NO: 14 for haPB, under SEQ ID NO: 15 and SEQ ID NO: 16 for KATA2A-haPB-TAF3, under SEQ ID NO: 29 and SEQ ID NO: 30 for KATA2A-haPB, and under SEQ ID NO: 31 and SEQ ID NO: 32 for haPB-TAF3.

FIG. 3: Maps of PBGGPEx2.0p_hc_PiggyBG and PBGGPEx2.0m_lc_PiggyBG. Promoter regions are shown as blue blocks: EF2/CMV hybrid promoter=strong heavy chain promoter, CMV/EF1 hybrid promoter=strong light chain promoter. Polyadenylation signals=pA are shown as yellow boxes. Antibiotic resistance genes, selection marker genes and the coding region for the light chain gene or rather the heavy chain gene are shown as orange arrows: pac=puromycin-N-acetyltransferase; dhfr=dehydrofolate reductase; aph=kanamycin resistance.

FIG. 4: IgG antibody concentrations of CHO-DG44 clones pools generated with different PiggyBac fusion proteins.

FIG. 5: IgG antibody titer concentrations of CHO-DG44 clones pools generated with different hyperactive PiggyBac fusion proteins.

FIG. 6: A: IgG antibody titer concentrations of CHO-DG44 clones pools generated with or without different hyperactive PiggyBac transposases. B: Real-Time PCR strategy to analyze and discriminate between total transgene copy number and randomly integrated transgenes. Gray arrows=PCR to detect randomly integrated transgenes. White arrows: PCR to detect transgene copies originating from random and transposase-mediated integration. C: Real-Time PCR results. Total and randomly integrated transgene copy numbers of samples derived from the hyperactive transposase or the hyperactive fusion domain variant TAF3-haPB relative to a sample generated without transposases.

### EXAMPLES

The examples given below are for illustrative purposes only and do not limit the invention described above in any way.

### Example 1

### Gene Optimization and Synthesis

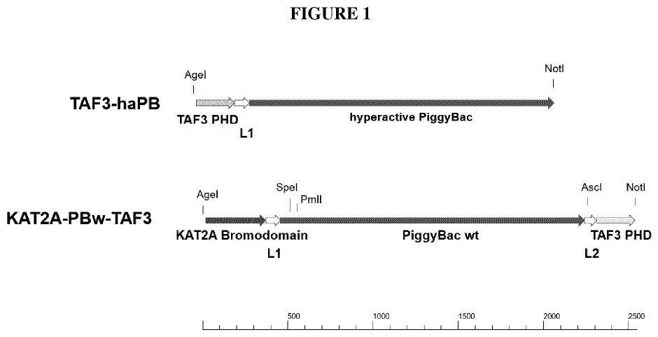

The amino acid sequences of PiggyBac wt transposase (Trichoplusia ni; GenBank accession number #AAA87375.2; SEQ ID NO: 6 [Virology 172(1) 156-169 1989]), a hyperactive PiggyBac transposase (I30V; G165S; M282V; N538K compared to PiggyBac wt transposase; SEQ ID NO: 6), TafIID sub III PHD domain (Homo sapiens; GenBank accession number #NP_114129.1 855 . . . 929; SEQ ID NO 20), histone acetyltransferase KAT2A Bromodomain (Homo sapiens; GenBank accession number NP_066564.2 741 . . . 837; SEQ ID NO 21), and two peptide linkers (linked: KLGGGAPAVGGGPKKLGGGAPAVGGGPK SEQ ID NO: 22; linker2: AAAKLGGGAPAVGGGPKAADKGAA SEQ ID NO: 23 were reverse translated and the resulting nucleotide sequences were linked as shown in FIG. 1.

The nucleotide sequences were optimized by knockout of cryptic splice sites and RNA destabilizing sequence elements, optimized for increased RNA stability and adapted to match the requirements of CHO cells (Cricetulus griseus) regarding the codon usage. The nucleotide sequences were synthesized by GeneArt Gene Synthesis (Life technologies). The coding sequence (CDS) of Taf3-haPB is shown under SEQ ID NO: 1 and the coding sequence (CDS) of KATA2A-PBw-TAF3 is shown under SEQ ID NO: 3. SEQ ID NO: 2 shows the amino acid sequence of Taf3-haPB and SEQ ID NO: 4 shows the amino acid sequence of KATA2A-PBw-TAF3.

### Example 2

### Construction of the Transposase Expression Plasmids

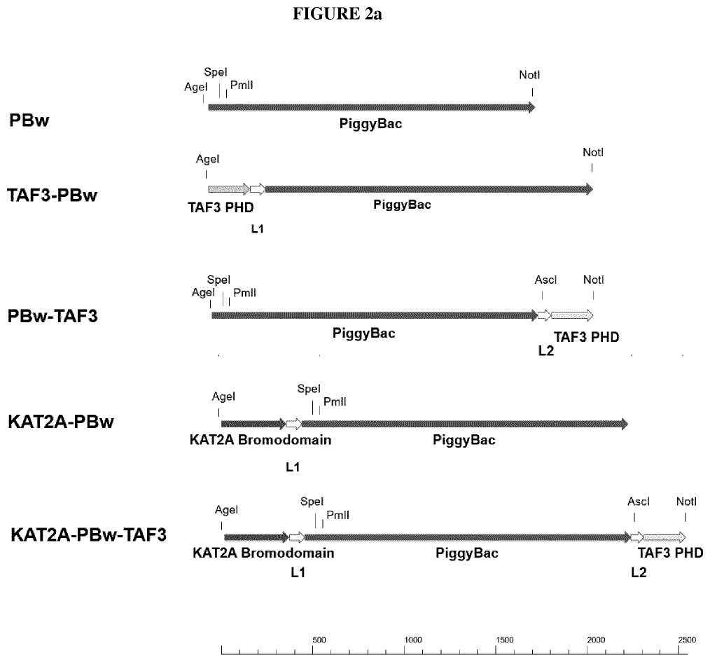

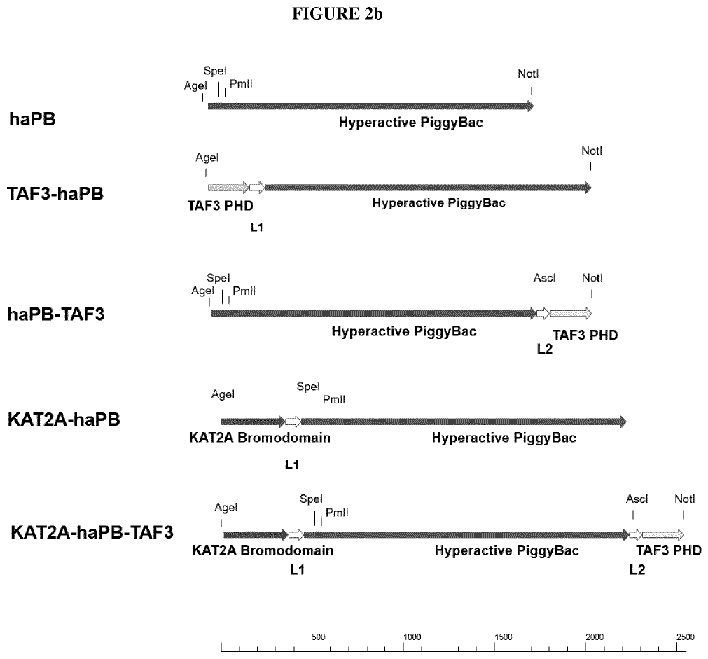

The synthesized constructs were used to generate the constructs shown in FIG. 2a and FIG. 2b using standard cloning procedures. The nucleotide sequences of the generated constructs are listed here under SEQ ID NO: 3 (KATA2A-PBw-TAF3), SEQ ID NO: 5 (PBw), SEQ ID NO: 7 (TAF3-PBw), SEQ ID NO: 9 (PBw-TAF3), SEQ ID NO: 11 (KAT2A-PBw), SEQ ID NO: 1 (Taf3-haPB), SEQ ID NO: 13 (haPB), SEQ ID NO: 15 (KATA2A-haPB-TAF3), SEQ ID NO: 29 (KATA2A-haPB) and SEQ ID NO: 31 (haPB-TAF3). The constructs were ligated into an expression vector, which allows transient expression of the transposase variants under control of the CMV promoter. General procedures for constructing expression plasmids are described in Sambrook, J., E. F. Fritsch and T. Maniatis: Cloning I/II/III, A Laboratory Manual New York/Cold Spring Harbor Laboratory Press, 1989, Second Edition.

### Example 3

### Construction of the Transposon Plasmids

Transposons were created containing the PiggyBac ITRs recognized by the PiggyBac transposase. Minimal ITR sequences of the PiggyBac transposon were integrated in the empty expression vectors PBGGPEx2.0m and PBGGPEx2.0p in 5′ and 3′ position to the bacterial backbone sequence with bacterial replication origin and antibiotic resistance gene by amplifying said bacterial backbone using the primers V1028_Piggy_forward, V1029_Piggy_reverse and V1036 Pbac_reverse 2 listed here under SEQ ID NO: 17 (V1028_Piggy_forward) and SEQ ID NO: 18 (V1029_Piggy_reverse) or rather SEQ ID NO: 17 (V1028_Piggy_forward) and SEQ ID NO: 19 (V1036 Pbac_reverse 2) and replacing the backbone of the corresponding vectors by one of the PCR-products via restriction digest with NdeI+NheI (PBGGPEx2.0m) or rather SfiI+NheI (PBGGPEx2.0p) to generate PBGGPEx2.0p_PiggyBG and PBGGPEx2.0m_PiggyBG.

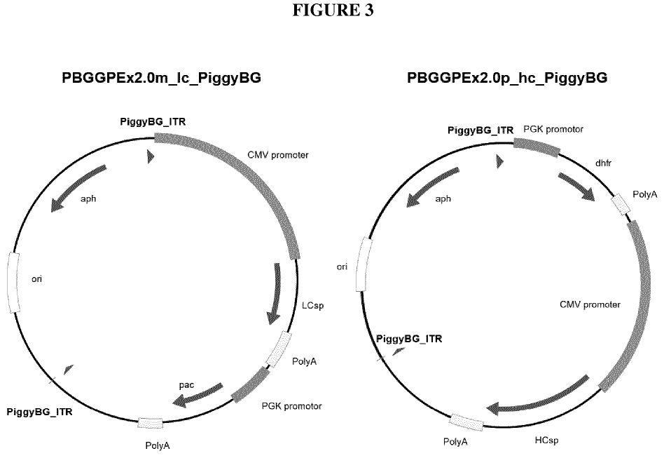

Synthetic heavy or rather light chain fragments of an monoclonal antibody assembled with a signal peptide were ligated into the transposon containing empty expression vectors PBGGPEx2.0p_PiggyBG and PBGGPEx2.0m_PiggyBG to generate PBGGPEx2.0p_hc_PiggyBG and PBGGPEx2.0m_lc_PiggyBG (FIG. 3). General procedures for constructing expression plasmids are described in Sambrook, J., E. F. Fritsch and T. Maniatis: Cloning I/II/III, A Laboratory Manual New York/Cold Spring Harbor Laboratory Press, 1989, Second Edition.

### Example 4

### Generation and Analysis of Clone Pools

As starter cell line the dihydrofolate reductase-deficient CHO cell line, CHO/DG44 [Urlaub et al., 1986, Proc Natl Acad Sci USA. 83 (2): 337-341] was used. The cell line was maintained in serum-free medium. Plasmids containing the PB transposons (PBGGPEx2.0p_hc_PiggyBG and PBGGPEx2.0m_lc_PiggyBG) and transient expression vectors for expression of one of the transposase variants each were transfected by electroporation according to the manufacturer's instructions (Neon Transfection System, Thermo Fisher Scientific). In each transfection 1.5 μg of circular HC and LC transposon vector DNA and 1.2 μg of circular transposase DNA were used. Transfectants were subjected to selection with puromycin and methotrexate to eliminate untransfected cells, as well as non- and low-producer. Two consecutive series of transfections and selections were performed using the same vector combinations, DNA amounts and selection conditions. After a selection period of two weeks selection pressure was removed and resulting clone pools were subjected to Fed-batch processes under generic conditions with defined seeding cell densities. Fed batch processes were performed in shake flasks (SF125, Corning) with working volumes of 30 mL in chemically defined culture medium. A chemically defined feed was applied every two days following a generic feeding regiment. Antibody concentrations of cell culture supernatant samples were determined by the Octet® RED96 System (Fortebio) against purified material of the expressed antibody as standard curve.

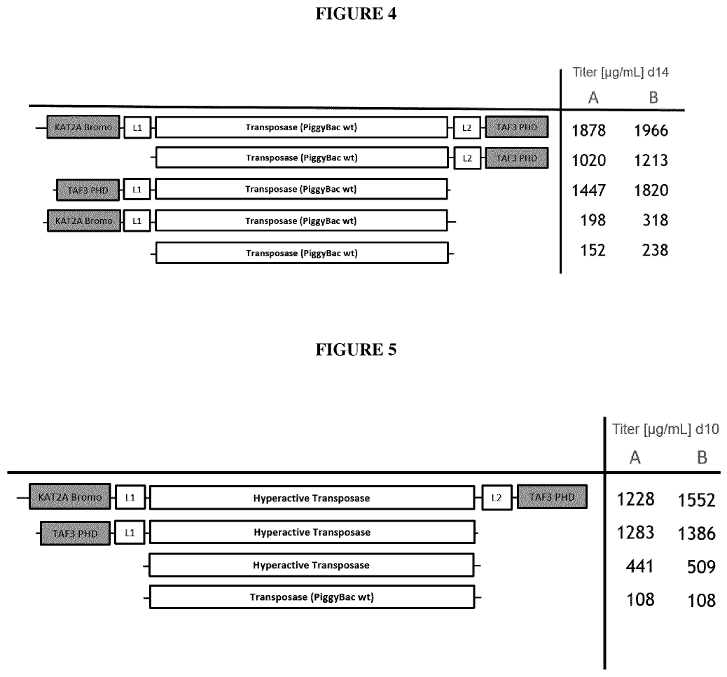

FIG. 4 shows the fed batch results of clone pools derived from wt PiggyBac transposase and wt PiggyBac fusion variants. For the clone pools generated with the KAT2A-PBw, TAF3-PBw, PBw-TAF3 and KAT2A-PBw-TAF3 fusions variants and wt PiggyBac transposase antibody yields were determined at day 14 of the fed-batch process. The strongest increase by a single chromatin reader was observed for TAF3 fused to the N terminus (TAF3-PBw: 8.4 fold based the arithmetic mean of the respective pools) and somewhat less when fused to the C terminus (PBw-TAF3: 5.7 fold) A very moderate increase (1.3 fold) was observed with KAT2A (KAT2A-PBw) fused in the same way. The addition of a second chromatin reader domain (in this case KAT2A) is supportive: pools generated with KAT2A-PBw-TAF3 show 1.7 higher expression compared to PBw-TAF3.

FIG. 5 shows the effects of the different fusion domains on a hyperactive (ha) transposase. Clone pools derived from this transposase achieved a ˜5.1-fold higher antibody concentration than the wt PiggyBac transposase pools. Compared to the hyperactive PiggyBac transposase pools antibody yields of the KAT2A-haPB, TAF3-haPB, haPB-TAF3 and KAT2A-haPB-TAF3 fusion variant clone pools were found to be ˜2-fold, ˜2.8-fold, ˜2.4-fold and 2.9-fold higher. Consequently, chromatin reader domains not only promote expression from cassettes introduced with the wt transposase but also for a hyperactive form. Remarkably, the fusion domains did not only improve both, the wt PiggyBag and hyperactive PiggyBac transposases, but expression levels are highly similar independent of the activity of the naked transposase

### Example 5

### Transposase Specific Genomic Integration of the Transposons.

Despite presence of a transposase expression unit in the transfection mix, the circular plasmid containing the transposon can also integrate into the host genome in an transposase-independent fashion. In this case, the plasmid is linearized at random and backbone as well as transposon sequence are integrated. In contrast, transposases mediate integration of the transposon sequences only. The frequency of transposase independent integration is rather similar between transfections carried out under identical transfection and selection conditions and can serve as an internal standard. For such random integration of the whole plasmid, segments located entirely within the transposon and segments reaching into the plasmid backbone are equally abundant. In pools generated in the presence of any transposase, transposon sequences will be more abundant. The ratio of pure transposon segments (transposase mediated and random integration events) and segments reaching into the backbone (random integration events) is a measure of transposase activity.

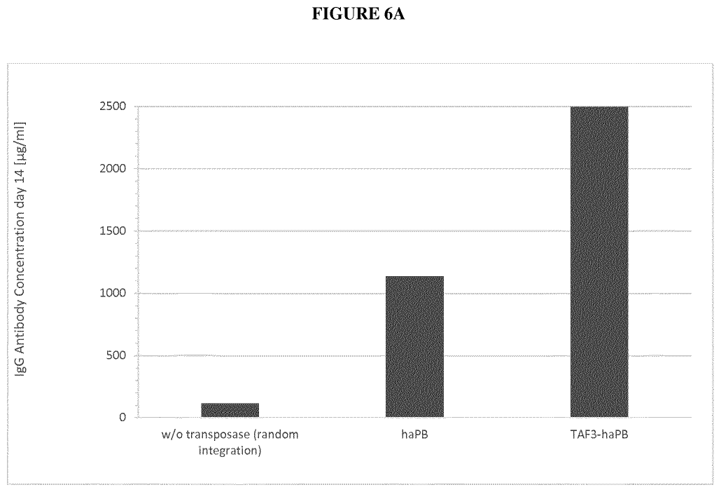

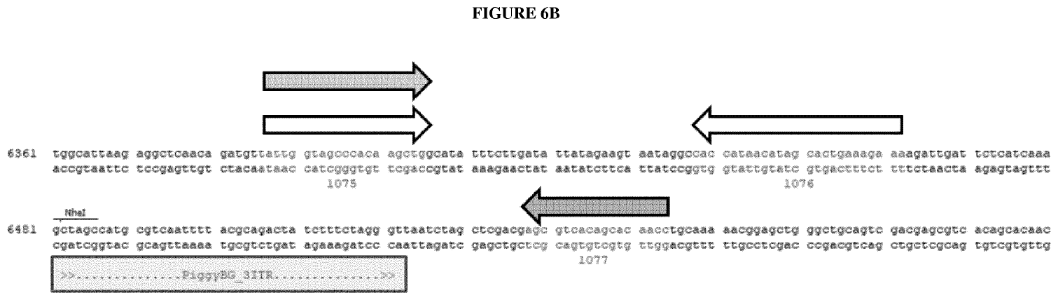

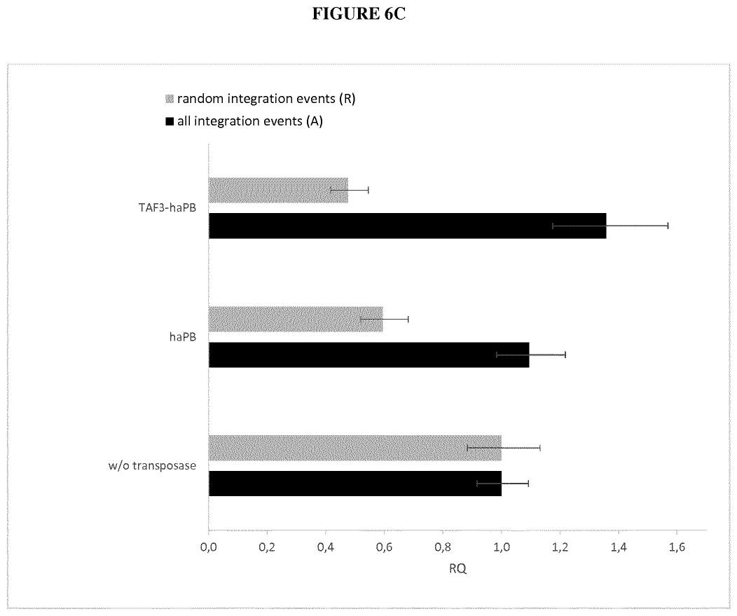

Genomic integration of the transposons was analysed by Real-Time qPCR. For sample preparation clone pools were generated and analysed in fed batch processes as described in Example 4, except for the DNA amounts. 7 μg of transposon vector DNA and 2.8 μg of transposase vector DNA was transfected. An additional clone pool was generated with circular transposon vectors only. For each clone pool genomic DNA was purified from 2E6 viable cells using the QIAamp DNA Blood Mini Kit (QIAGEN, REF: 51104) and DNA Purification from Blood or Body Fluids, Spin Protocol. Genomic DNA concentrations were determined by a NanoPhotometer NP80 (Implen) and genomic DNA samples were diluted to a concentration of 10 ng/μl with DEPC Treated Water (Invitrogen, REF: 46-2224). The PCR reaction mixes were prepared as follows: 90 nM forward primer, 90 nM backwards primer, 50 ng sample DNA, 10 μL Power SYBR Green PCR Master Mix (Applied Biosystems, REF: 4367659), add to 20 μL with DEPC Treated Water (Invitrogen, REF: 46-2224). Samples were analyzed as triplicates using a StepOnePlus Real-Time PCR System (Applied Biosystems). Three different primer sets and PCR reactions were performed for each sample. To measure the ration of specific integrated transposons and random integrated plasmid DNA the primers V1075 PBG forward (TATTGGTAGCCCACAAGCTG; SEQ ID NO: 26) and V1076 PBG reverse 1 (TTTCTTTCAGTGCTATGTTATGGTG; SEQ ID NO: 27) or rather V1075 PBG forward (TATTGGTAGCCCACAAGCTG; SEQ ID NO: 26) and V1077 PBG reverse 2 (GGTTGTGCTGTGACGCT; (SEQ ID NO: 28) were used to amplify a small fragment within the transposon (77 bp fragment, specific for integration of transposon and random integration of plasmid DNA) or rather a fragment comprising the 5′ PiggyBac ITR (169 bp fragment, specific for random integration of plasmid DNA) (FIG. 6). In order to normalize and compare the different samples the primer V455 qPCR-ALU-Forward (TAAgAgCACCAACTgCTCTTCCA; SEQ ID NO: 33) and V456 qPCR-ALU-Reverse (ACCAgAAgAgggCACCAgATCT; SEQ ID NO: 34) were used to amplify an endogenous ALU sequence. The following PCR conditions were applied: 95° C. for 10 min, 95° C. for 15 sec, 60° C. for 60 sec, 40 cycles. Real time PCR data were analysed using the comparative CT(ΔΔCT) method.

3 pools were compared: the first generated with transposase, the second with the same transposase fused to the TAF3 domain (TAF3-haPB) and a third without any transposase. In the fed batch processes titers of 1100 μg/ml, 2500 μg/ml and 115 μg/ml were measured respectively as shown in FIG. 6A.

Using the Real-Time PCR detection strategy shown in FIG. 6B, genomic DNA samples of the three clone pool were analysed for relative copy numbers of the transposon-specific segment (all integration events (A)—transposase-mediated and random) and a segment containing both transposon and backbone sequences (random only (R)) as outlined in FIG. 6C. Relative copy number of the transposase-mediated integration (T) can be calculated as A−R=T

In the absence of transposase A=R and T=0. Hence, relative copy numbers determined for both R and A were set to 1 to account for different length PCR fragments.

In the presence of any transposase A>>R, a ratio of transposase dependent to random integration can be determined. For the transposase without a fusion domain this ratio is T/R=A−R/R=0.84. Although under the given conditions random integration still dominates slightly in terms of copy number, expression from the respective pools is considerably higher showing the benefit of the transposase approach. This may be due to removal of prokaryotic backbone sequences next to the transgenes and selection of active loci by the transposase itself. For the transposase with the TAF3 fusion domain this ratio is T/R=A−R/R=1.86. Here, the transposase-dependent integration events dominate. Respective cells benefit from the higher expression of the selection marker genes compared to the random approach which results in earlier recovery and multiplication during selection at the expense of cells harbouring randomly integrated copies. In addition, the titer obtained with this pool is 2.5× higher compared to that obtained with the unmodified transposase. Strikingly, chromatin reader domain can clearly potentiate stringency of selection for highly active sites on the background of such selection by the transposase itself.

1. A polypeptide comprising a transposase or a fragment or a derivative thereof having transposase function and at least one heterologous chromatin reader element (CRE).

2-3. (canceled)

4. The polypeptide of claim 1, wherein the at least one heterologous CRE is a chromatin reader domain (CRD).

5. The polypeptide of claim 4, wherein the at least one heterologous CRD is a naturally occurring CRD recognizing histone methylation degree and/or acetylation state of histones.

6. (canceled)

7. The polypeptide of claim 5, wherein the naturally occurring CRD recognising histone methylation degree is a plant homeodomain (PHD) type zinc finger, or the naturally occurring CRD regonizing the acetylation state of histones is a bromodomain.

8. The polypeptide of claim 7, wherein the PHD type zinc finger is a transcription initiation factor TFIID subunit 3 PHD, or the bromodomain is a histone acetyltransferese KAT2A domain.

9. The polypeptide of claim 8, wherein the transcription initiation factor TFIID subunit 3 PHD has an amino acid sequence according to SEQ ID NO: 20, or
the histone acetyltransferase KAT2A domain has an amino acid sequence according to SEQ ID No. 21.

10-12. (canceled)

13. The polypeptide of claim 1, wherein the CRE is an artificial CRE recognizing histone tails with specific methylated and/or acetylated sites.

14. (canceled)

15. The polypeptide of claim 13, wherein the artificial CRE is selected from the group consisting of a micro antibody, a single chain antibody, an antibody fragment, an affibody, an affilin, an anticalin, an atrimer, a DARPin, a FN2 scaffold, a fynomer, and a Kunitz domain.

16. The polypeptide of claim 1, wherein the transposase is selected from the group consisting of a wild-type PiggyBac transposase, a hyperactive PiggyBac transposase, a wild-type PiggyBac-like transposase, a hyperactive PiggyBac-like transposase, a sleeping beauty transposase, and a Tol2 transposase.

17-19. (canceled)

20. A polynucleotide encoding the polypeptide of claim 1.

21. A vector comprising the polynucleotide of claim 20.

22. A method for producing a transgenic cell comprising the steps of:
(i) providing a cell, and
(ii) introducing
a transposable element comprising at least one polynucleotide of interest, and
a polypeptide of
claim 1
into the cell, thereby producing the transgenic cell.

23-25. (canceled)

26. The method of claim 22, wherein the transposable element comprises terminal repeats (TRs) and wherein the at least one polynucleotide of interest is flanked by these TRs.

27. (canceled)

28. The method of claim 22, wherein the transposable element is a DNA transposable element, or
a retrotransposable element.

29. The method of claim 28, wherein the DNA transposable element comprises inverted terminal repeats (ITRs), or the retrotransposable element is a long terminal repeat (LTR) retrotransposable element.

30-32. (canceled)

33. The method of claim 22, wherein the cell is a eukaryotic cell.

34-35. (canceled)

36. The method of claim 22, wherein the at least one polynucleotide of interest is selected from the group consisting of a polynucleotide encoding a polypeptide, a non-coding polynucleotide, a polynucleotide comprising a promoter sequence, a polynucleotide encoding a mRNA, a polynucleotide encoding a tag, and a viral polynucleotide.

37-38. (canceled)

39. A kit comprising
(i) a transposable element comprising a cloning site for inserting at least one polynucleotide of interest, and
(ii) a polypeptide of claim 1.

40-50. (canceled)

51. A targeting system comprising
(i) a transposable element comprising at least one polynucleotide of interest, and
(ii) a polypeptide of claim 1.

52-54. (canceled)

55. A method for producing a transgenic cell comprising the steps of:
providing a cell, and
(ii) introducing
a transposable element comprising at least one polynucleotide of interest, and
a polynucleotide of claim 20
into the cell, thereby producing the transgenic cell.

56. A method for producing a transgenic cell comprising the steps of:
(i) providing a cell, and
(ii) introducing
a transposable element comprising at least one polynucleotide of interest, and a vector of claim 21
into the cell, thereby producing the transgenic cell.

57. A kit comprising
(i) a transposable element comprising a cloning site for inserting at least one polynucleotide of interest, and
(ii) a polynucleotide of claim 20.

58. A kit comprising
(i) a transposable element comprising a cloning site for inserting at least one polynucleotide of interest, and
(ii) a vector of claim 21.

59. A kit comprising
(i) a transposable element comprising a cloning site for inserting at least one polynucleotide of interest, and
(ii) at least one heterologous CRE and a polypeptide comprising a transposase or a fragment or a derivative thereof having transposase function.

60. A targeting system comprising
(i) a transposable element comprising at least one polynucleotide of interest, and
(ii) a polynucleotide of claim 20.

61. A targeting system comprising
(i) a transposable element comprising at least one polynucleotide of interest, and
(ii) a vector of claim 21.

62. A targeting system comprising
(i) a transposable element comprising at least one polynucleotide of interest,
(ii) at least one heterologous CRE, optionally associated with the transposable element, and
(iii) a polypeptide comprising a transposase or a fragment or a derivative thereof having transposase function.


SEQUENCE LISTING (34 sequences)

SEQ ID NO: 1 - DNA, 2120 bp, Artificial Sequence
  Taf3-haPB
  CDS (16)..(2109)
accggtggatccggcatggtcatcagagatgagtggggcaatcagatctggatctgtcccggctgcaacaagcctgacgacggctctcctatgatcggctgcgacgactgtgacgactggtatcactggccttgcgtgggcatcatgaccgctccacctgaagagatgcagtggttctgccccaagtgcgccaacaagaagaaggataagaagcacaagaagcggaagcacagagcccacaagcttggaggtggtgctcctgctgttggcggcggacctaaaaaacttggaggcggagcaccagctgtcggcggaggtcctaaagccatgggatcttctctggacgacgagcacatcctgtctgccctgctgcagtctgacgatgaactcgtgggcgaagattccgactccgaggtgtccgaccatgtgtctgaggacgacgtgcagtccgataccgaggaagccttcatcgacgaggtgcacgaagtgcagcctacctcttccggctctgagatcctggacgagcagaacgtgatcgagcagcctggatcttccctggcctccaacagaatcctgacactgcctcagcggaccatccggggcaagaacaagcactgctggtccacctctaagagcacccggcggtctagagtgtccgctctgaatattgtgcggtcccagaggggccccaccagaatgtgccggaacatctacgaccctctgctgtgcttcaagctgttcttcaccgacgagatcatctccgagatcgtgaagtggaccaacgccgagatctctctgaagcggcgcgagtctatgacctctgccaccttccgggacaccaacgaggatgagatctacgccttcttcggcatcctggtcatgacagccgtgcggaaggacaaccacatgtccaccgacgacctgttcgacagatccctgtccatggtgtacgtgtccgtgatgtccagggacagattcgacttcctgatccggtgcctgcggatggacgacaagtctatcagacccacactgcgcgagaacgacgtgttcacacctgtgcggaagatctgggacctgttcatccaccagtgcatccagaactacacccctggcgctcacctgaccatcgacgaacagctgctgggcttcagaggcagatgccctttccgggtgtacatccccaacaagccctctaagtacggcatcaagatcctgatgatgtgcgactccggcaccaagtacatgatcaacggcatgccctacctcggcagaggcacccaaacaaatggcgtgccactgggcgagtactacgtgaaagaactgtccaagcctgtgcacggctcctgcagaaacatcacctgtgataactggttcacctccattcctctggccaagaacctgctgcaagagccttacaagctgacaatcgtgggcaccgtgcggtccaacaagcgggaaattcctgaggtgctgaagaactctcggtccagacctgtgggcacctccatgttctgtttcgacggccctctgacactggtgtcctacaagcctaagcctgccaagatggtgtacctgctgtcctcctgtgacgaggacgccagcatcaatgagtccaccggcaagccccagatggtcatgtactacaaccagaccaaaggcggcgtggacaccctggaccagatgtgctctgtgatgacctgctccagaaagaccaacagatggcccatggctctgctgtacggcatgatcaatatcgcctgcatcaacagcttcatcatctactcccacaacgtgtcctccaagggcgagaaggtgcagtcccggaaaaagttcatgcggaacctgtatatgtccctgacctccagcttcatgagaaagcggctggaagcccctacactgaagcgctacctgcgggacaacatctccaacatcctgcctaaagaggtgcccggcaccagcgacgactctacagaggaacccgtgatgaagaagaggacctactgcacctactgtccctccaagatccggcggaaggccaacgcctcttgcaaaaagtgcaagaaagtgatctgccgcgagcacaacatcgatatgtgccagtcctgcttctgagcggccgc

SEQ ID NO: 2 - protein, 698 aa, Artificial Sequence
  Synthetic Construct
MVIRDEWGNQIWICPGCNKPDDGSPMIGCDDCDDWYHWPCVGIMTAPPEEMQWFCPKCANKKKDKKHKKRKHRAHKLGGGAPAVGGGPKKLGGGAPAVGGGPKAMGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 3 - DNA, 2546 bp, Artificial Sequence
  KAT2A-PBw-Taf3
  CDS (16)..(2538)
accggtggatccggcatgaaggaaaagggcaaagagctgaaggaccccgaccagctgtacaccacactgaagaatctgctggcccagatcaagtctcacccctccgcctggcctttcatggaacccgtgaagaagtctgaggcccctgactactacgaagtgatcagattccccatcgacctcaagaccatgaccgagcggctgagatcccggtactacgtgaccagaaagctgttcgtggccgacctgcagagagtgatcgccaactgtagagagtacaaccctcctgactccgagtactgcagatgcgcctccgctctggaaaagttcttctacttcaagctgaaagaaggcggcctgatcgacaagaagcttggaggcggagcaccagctgttggcggaggacctaaaaaactcggaggtggcgctcctgctgtcggaggcggacctaaagctatgggcagctctctggacgacgagcacatcctgtctgccctgctgcagtccgacgatgaactagtgggcgaagattccgactccgagatctccgatcacgtgtccgaggacgacgtgcagtctgataccgaggaagccttcatcgacgaggtgcacgaagtgcagcctacctcttccggctctgagatcctggacgagcagaacgtgatcgagcagcctggatcctctctggcctccaacagaatcctgacactgccccagagaaccatccggggcaagaacaagcactgctggtccacctccaagtctacccggcggtctagagtgtccgctctgaatattgtgcggtcccagaggggccccaccagaatgtgccggaacatctacgaccctctgctgtgtttcaagctgttcttcaccgacgagatcatcagcgagatcgtgaagtggaccaacgccgagatcagcctgaagcggcgggaatctatgaccggcgccaccttcagagacaccaacgaggatgagatctacgccttcttcggcatcctggtcatgacagccgtgcggaaggacaaccacatgtccaccgacgacctgttcgacagatccctgtccatggtgtacgtgtccgtgatgagccgggacagattcgacttcctgatccggtgcctgcggatggacgacaagtccatcagacccacactgcgcgagaacgacgtgttcacacctgtgcggaagatctgggacctgttcatccaccagtgcatccagaactacacccctggcgctcacctgaccatcgatgaacagctgctgggcttcagaggcagatgccccttcagaatgtacatccccaacaagccctctaagtacggcatcaagatcctgatgatgtgcgactccggcaccaagtacatgatcaacggcatgccctacctcggcagaggcacccaaacaaatggcgtgccactgggcgagtactatgtgaaagaactgtccaagcctgtgcacggctcctgcagaaacatcacctgtgacaactggttcaccagcattcctctggccaagaacctgctgcaagagccctacaagctgacaatcgtgggcaccgtgcggtccaacaagcgggaaattcctgaggtgctgaagaactctcggtccagacctgtgggcacctccatgttctgtttcgacggccctctgacactggtgtcctacaagcctaagcctgccaagatggtgtacctgctgtcctcctgtgacgaggacgccagcatcaatgagtccaccggcaagccccagatggtcatgtactacaaccagaccaaaggcggcgtggacaccctggaccagatgtgctctgtgatgacctgctccagaaagaccaacagatggcccatggctctgctgtacggcatgatcaatatcgcctgcatcaacagcttcatcatctactcccacaacgtgtcctccaagggcgagaaggtgcagtcccggaagaaattcatgcggaacctgtatatgtccctgacctccagcttcatgagaaagcggctggaagcccctactctgaagagatacctgcgggacaacatctccaacatcctgcctaacgaggtgcccggcaccagcgacgattctacagaggaacctgtgatgaagaagcggacctactgcacctactgtccctccaagatccggcggaaggccaacgcctcttgcaaaaagtgcaagaaagtgatctgccgcgagcacaacatcgacatgtgccagtcttgtttcgccgctgctaaacttggtggtggcgcgccggcagtcggcggaggtccaaaagctgctgataagggcgctgccgtgatcagagatgagtggggcaatcagatctggatctgtcctggctgcaacaagcctgacgacggctctcctatgatcggctgcgacgactgtgacgattggtatcactggccctgcgtgggcatcatgaccgctccacctgaagaaatgcagtggttctgccccaagtgcgccaacaagaagaaggataagaagcacaagaagcgcaagcacagggcccactgatgagcggccgc

SEQ ID NO: 4 - protein, 839 aa, Artificial Sequence
  Synthetic Construct
MKEKGKELKDPDQLYTTLKNLLAQIKSHPSAWPFMEPVKKSEAPDYYEVIRFPIDLKTMTERLRSRYYVTRKLFVADLQRVIANCREYNPPDSEYCRCASALEKFFYFKLKEGGLIDKKLGGGAPAVGGGPKKLGGGAPAVGGGPKAMGSSLDDEHILSALLQSDDELVGEDSDSEISDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTGATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRMYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPNEVPGTSDDSTEEPVMKKRTYCTYCPSKIRRKANASCKKCKKVICREHNIDMCQSCFAAAKLGGGAPAVGGGPKAADKGAAVIRDEWGNQIWICPGCNKPDDGSPMIGCDDCDDWYHWPCVGIMTAPPEEMQWFCPKCANKKKDKKHKKRKHRAH

SEQ ID NO: 5 - DNA, 1807 bp, Artificial Sequence
  PBw
  CDS (12)..(1799)
accggtccggcatgggctctagcctggacgacgagcacattctgtctgccctgctgcagtccgacgatgaactcgtgggcgaagattccgactccgagatctctgaccacgtgtccgaggacgacgtgcagtctgataccgaggaagccttcatcgacgaggtgcacgaagtgcagcctacctcttccggctctgagatcctggacgagcagaacgtgatcgagcagcctggatcctctctggcctccaacagaatcctgacactgccccagagaaccatccggggcaagaacaagcactgctggtccacctccaagtctacccggcggtctagagtgtccgctctgaatattgtgcggtcccagaggggccccaccagaatgtgccggaacatctacgaccctctgctgtgtttcaagctgttcttcaccgacgagatcatcagcgagatcgtgaagtggaccaacgccgagatcagcctgaagcggcgggaatctatgaccggcgccaccttcagagacaccaacgaggatgagatctacgccttcttcggcatcctggtcatgacagccgtgcggaaggacaaccacatgtccaccgacgacctgttcgacagatccctgtccatggtgtacgtgtccgtgatgagccgggacagattcgacttcctgatccggtgcctgcggatggacgacaagtccatcagacccacactgcgcgagaacgacgtgttcacacctgtgcggaagatctgggacctgttcatccaccagtgcatccagaactacacccctggcgctcacctgaccatcgatgaacagctgctgggcttcagaggcagatgccccttcagaatgtacatccccaacaagccctctaagtacggcatcaagatcctgatgatgtgcgactccggcaccaagtacatgatcaacggcatgccctacctcggcagaggcacccaaacaaatggcgtgccactgggcgagtactatgtgaaagaactgtccaagcctgtgcacggctcctgcagaaacatcacctgtgacaactggttcaccagcattcctctggccaagaacctgctgcaagagccctacaagctgacaatcgtgggcaccgtgcggtccaacaagcgggaaattcctgaggtgctgaagaactctcggtccagacctgtgggcacctccatgttctgtttcgacggccctctgacactggtgtcctacaagcctaagcctgccaagatggtgtacctgctgtcctcctgtgacgaggacgccagcatcaatgagtccaccggcaagccccagatggtcatgtactacaaccagaccaaaggcggcgtggacaccctggaccagatgtgctctgtgatgacctgctccagaaagaccaacagatggcccatggctctgctgtacggcatgatcaatatcgcctgcatcaacagcttcatcatctactcccacaacgtgtcctccaagggcgagaaggtgcagtcccggaagaaattcatgcggaacctgtatatgtccctgacctccagcttcatgagaaagcggctggaagcccctactctgaagagatacctgcgggacaacatctccaacatcctgcctaacgaggtgcccggcaccagcgacgattctacagaggaacctgtgatgaagaagcggacctactgcacctactgtccctccaagatccggcggaaggccaacgcctcttgcaaaaagtgcaagaaagtgatctgccgcgagcacaacatcgacatgtgccagtcttgtttctgatgagcggccgc

SEQ ID NO: 6 - protein, 594 aa, Artificial Sequence
  Synthetic Construct
MGSSLDDEHILSALLQSDDELVGEDSDSEISDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTGATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRMYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPNEVPGTSDDSTEEPVMKKRTYCTYCPSKIRRKANASCKKCKKVICREHNIDMCQSCF

SEQ ID NO: 7 - DNA, 2123 bp, Artificial Sequence
  Taf3-PBw
  CDS (16)..(2115)
accggtggatccggcatggtcatcagagatgagtggggcaatcagatctggatctgtcccggctgcaacaagcctgacgacggctctcctatgatcggctgcgacgactgtgacgactggtatcactggccttgcgtgggcatcatgaccgctccacctgaagagatgcagtggttctgccccaagtgcgccaacaagaagaaggataagaagcacaagaagcggaagcacagggcccacaaacttggaggtggtgctcctgctgttggcggcggacctaaaaaacttggtggcggagcaccagctgtcggcggaggtcctaaagccatgggctctagcctggacgacgagcacattctgtctgccctgctgcagtccgacgatgaactcgtgggcgaagattccgactccgagatctctgaccacgtgtccgaggacgacgtgcagtctgataccgaggaagccttcatcgacgaggtgcacgaagtgcagcctacctcttccggctctgagatcctggacgagcagaacgtgatcgagcagcctggatcctctctggcctccaacagaatcctgacactgccccagagaaccatccggggcaagaacaagcactgctggtccacctccaagtctacccggcggtctagagtgtccgctctgaatattgtgcggtcccagaggggccccaccagaatgtgccggaacatctacgaccctctgctgtgtttcaagctgttcttcaccgacgagatcatcagcgagatcgtgaagtggaccaacgccgagatcagcctgaagcggcgggaatctatgaccggcgccaccttcagagacaccaacgaggatgagatctacgccttcttcggcatcctggtcatgacagccgtgcggaaggacaaccacatgtccaccgacgacctgttcgacagatccctgtccatggtgtacgtgtccgtgatgagccgggacagattcgacttcctgatccggtgcctgcggatggacgacaagtccatcagacccacactgcgcgagaacgacgtgttcacacctgtgcggaagatctgggacctgttcatccaccagtgcatccagaactacacccctggcgctcacctgaccatcgatgaacagctgctgggcttcagaggcagatgccccttcagaatgtacatccccaacaagccctctaagtacggcatcaagatcctgatgatgtgcgactccggcaccaagtacatgatcaacggcatgccctacctcggcagaggcacccaaacaaatggcgtgccactgggcgagtactatgtgaaagaactgtccaagcctgtgcacggctcctgcagaaacatcacctgtgacaactggttcaccagcattcctctggccaagaacctgctgcaagagccctacaagctgacaatcgtgggcaccgtgcggtccaacaagcgggaaattcctgaggtgctgaagaactctcggtccagacctgtgggcacctccatgttctgtttcgacggccctctgacactggtgtcctacaagcctaagcctgccaagatggtgtacctgctgtcctcctgtgacgaggacgccagcatcaatgagtccaccggcaagccccagatggtcatgtactacaaccagaccaaaggcggcgtggacaccctggaccagatgtgctctgtgatgacctgctccagaaagaccaacagatggcccatggctctgctgtacggcatgatcaatatcgcctgcatcaacagcttcatcatctactcccacaacgtgtcctccaagggcgagaaggtgcagtcccggaagaaattcatgcggaacctgtatatgtccctgacctccagcttcatgagaaagcggctggaagcccctactctgaagagatacctgcgggacaacatctccaacatcctgcctaacgaggtgcccggcaccagcgacgattctacagaggaacctgtgatgaagaagcggacctactgcacctactgtccctccaagatccggcggaaggccaacgcctcttgcaaaaagtgcaagaaagtgatctgccgcgagcacaacatcgacatgtgccagtcttgtttctgatgagcggccgc

SEQ ID NO: 8 - protein, 698 aa, Artificial Sequence
  Synthetic Construct
MVIRDEWGNQIWICPGCNKPDDGSPMIGCDDCDDWYHWPCVGIMTAPPEEMQWFCPKCANKKKDKKHKKRKHRAHKLGGGAPAVGGGPKKLGGGAPAVGGGPKAMGSSLDDEHILSALLQSDDELVGEDSDSEISDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTGATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRMYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPNEVPGTSDDSTEEPVMKKRTYCTYCPSKIRRKANASCKKCKKVICREHNIDMCQSCF

SEQ ID NO: 9 - DNA, 2104 bp, Artificial Sequence
  PBw-Taf3
  CDS (12)..(2093)
accggtccggcatgggctctagcctggacgacgagcacattctgtctgccctgctgcagtccgacgatgaactcgtgggcgaagattccgactccgagatctctgaccacgtgtccgaggacgacgtgcagtctgataccgaggaagccttcatcgacgaggtgcacgaagtgcagcctacctcttccggctctgagatcctggacgagcagaacgtgatcgagcagcctggatcctctctggcctccaacagaatcctgacactgccccagagaaccatccggggcaagaacaagcactgctggtccacctccaagtctacccggcggtctagagtgtccgctctgaatattgtgcggtcccagaggggccccaccagaatgtgccggaacatctacgaccctctgctgtgtttcaagctgttcttcaccgacgagatcatcagcgagatcgtgaagtggaccaacgccgagatcagcctgaagcggcgggaatctatgaccggcgccaccttcagagacaccaacgaggatgagatctacgccttcttcggcatcctggtcatgacagccgtgcggaaggacaaccacatgtccaccgacgacctgttcgacagatccctgtccatggtgtacgtgtccgtgatgagccgggacagattcgacttcctgatccggtgcctgcggatggacgacaagtccatcagacccacactgcgcgagaacgacgtgttcacacctgtgcggaagatctgggacctgttcatccaccagtgcatccagaactacacccctggcgctcacctgaccatcgatgaacagctgctgggcttcagaggcagatgccccttcagaatgtacatccccaacaagccctctaagtacggcatcaagatcctgatgatgtgcgactccggcaccaagtacatgatcaacggcatgccctacctcggcagaggcacccaaacaaatggcgtgccactgggcgagtactatgtgaaagaactgtccaagcctgtgcacggctcctgcagaaacatcacctgtgacaactggttcaccagcattcctctggccaagaacctgctgcaagagccctacaagctgacaatcgtgggcaccgtgcggtccaacaagcgggaaattcctgaggtgctgaagaactctcggtccagacctgtgggcacctccatgttctgtttcgacggccctctgacactggtgtcctacaagcctaagcctgccaagatggtgtacctgctgtcctcctgtgacgaggacgccagcatcaatgagtccaccggcaagccccagatggtcatgtactacaaccagaccaaaggcggcgtggacaccctggaccagatgtgctctgtgatgacctgctccagaaagaccaacagatggcccatggctctgctgtacggcatgatcaatatcgcctgcatcaacagcttcatcatctactcccacaacgtgtcctccaagggcgagaaggtgcagtcccggaagaaattcatgcggaacctgtatatgtccctgacctccagcttcatgagaaagcggctggaagcccctactctgaagagatacctgcgggacaacatctccaacatcctgcctaacgaggtgcccggcaccagcgacgattctacagaggaacctgtgatgaagaagcggacctactgcacctactgtccctccaagatccggcggaaggccaacgcctcttgcaaaaagtgcaagaaagtgatctgccgcgagcacaacatcgacatgtgccagtcttgtttcgccgctgctaaacttggtggtggcgcgccggcagtcggcggaggtccaaaagctgctgataagggcgctgccgtgatcagagatgagtggggcaatcagatctggatctgtcctggctgcaacaagcctgacgacggctctcctatgatcggctgcgacgactgtgacgattggtatcactggccctgcgtgggcatcatgaccgctccacctgaagaaatgcagtggttctgccccaagtgcgccaacaagaagaaggataagaagcacaagaagcgcaagcacagggcccactgatgagcggccgcgac

SEQ ID NO: 10 - protein, 692 aa, Artificial Sequence
  Synthetic Construct
MGSSLDDEHILSALLQSDDELVGEDSDSEISDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTGATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRMYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPNEVPGTSDDSTEEPVMKKRTYCTYCPSKIRRKANASCKKCKKVICREHNIDMCQSCFAAAKLGGGAPAVGGGPKAADKGAAVIRDEWGNQIWICPGCNKPDDGSPMIGCDDCDDWYHWPCVGIMTAPPEEMQWFCPKCANKKKDKKHKKRKHRAH

SEQ ID NO: 11 - DNA, 2252 bp, Artificial Sequence
  KAT2A-PBw
  CDS (16)..(2244)
accggtggatccggcatgaaggaaaagggcaaagagctgaaggaccccgaccagctgtacaccacactgaagaatctgctggcccagatcaagtctcacccctccgcctggcctttcatggaacccgtgaagaagtctgaggcccctgactactacgaagtgatcagattccccatcgacctcaagaccatgaccgagcggctgagatcccggtactacgtgaccagaaagctgttcgtggccgacctgcagagagtgatcgccaactgtagagagtacaaccctcctgactccgagtactgcagatgcgcctccgctctggaaaagttcttctacttcaagctgaaagaaggcggcctgatcgacaagaagcttggaggcggagcaccagctgttggcggaggacctaaaaaactcggaggtggcgctcctgctgtcggaggcggacctaaagctatgggcagctctctggacgacgagcacatcctgtctgccctgctgcagtccgacgatgaactagtgggcgaagattccgactccgagatctccgatcacgtgtccgaggacgacgtgcagtctgataccgaggaagccttcatcgacgaggtgcacgaagtgcagcctacctcttccggctctgagatcctggacgagcagaacgtgatcgagcagcctggatcctctctggcctccaacagaatcctgacactgccccagagaaccatccggggcaagaacaagcactgctggtccacctccaagtctacccggcggtctagagtgtccgctctgaatattgtgcggtcccagaggggccccaccagaatgtgccggaacatctacgaccctctgctgtgtttcaagctgttcttcaccgacgagatcatcagcgagatcgtgaagtggaccaacgccgagatcagcctgaagcggcgggaatctatgaccggcgccaccttcagagacaccaacgaggatgagatctacgccttcttcggcatcctggtcatgacagccgtgcggaaggacaaccacatgtccaccgacgacctgttcgacagatccctgtccatggtgtacgtgtccgtgatgagccgggacagattcgacttcctgatccggtgcctgcggatggacgacaagtccatcagacccacactgcgcgagaacgacgtgttcacacctgtgcggaagatctgggacctgttcatccaccagtgcatccagaactacacccctggcgctcacctgaccatcgatgaacagctgctgggcttcagaggcagatgccccttcagaatgtacatccccaacaagccctctaagtacggcatcaagatcctgatgatgtgcgactccggcaccaagtacatgatcaacggcatgccctacctcggcagaggcacccaaacaaatggcgtgccactgggcgagtactatgtgaaagaactgtccaagcctgtgcacggctcctgcagaaacatcacctgtgacaactggttcaccagcattcctctggccaagaacctgctgcaagagccctacaagctgacaatcgtgggcaccgtgcggtccaacaagcgggaaattcctgaggtgctgaagaactctcggtccagacctgtgggcacctccatgttctgtttcgacggccctctgacactggtgtcctacaagcctaagcctgccaagatggtgtacctgctgtcctcctgtgacgaggacgccagcatcaatgagtccaccggcaagccccagatggtcatgtactacaaccagaccaaaggcggcgtggacaccctggaccagatgtgctctgtgatgacctgctccagaaagaccaacagatggcccatggctctgctgtacggcatgatcaatatcgcctgcatcaacagcttcatcatctactcccacaacgtgtcctccaagggcgagaaggtgcagtcccggaagaaattcatgcggaacctgtatatgtccctgacctccagcttcatgagaaagcggctggaagcccctactctgaagagatacctgcgggacaacatctccaacatcctgcctaacgaggtgcccggcaccagcgacgattctacagaggaacctgtgatgaagaagcggacctactgcacctactgtccctccaagatccggcggaaggccaacgcctcttgcaaaaagtgcaagaaagtgatctgccgcgagcacaacatcgacatgtgccagtcttgtttctgatgagcggccgc

SEQ ID NO: 12 - protein, 741 aa, Artificial Sequence
  Synthetic Construct
MKEKGKELKDPDQLYTTLKNLLAQIKSHPSAWPFMEPVKKSEAPDYYEVIRFPIDLKTMTERLRSRYYVTRKLFVADLQRVIANCREYNPPDSEYCRCASALEKFFYFKLKEGGLIDKKLGGGAPAVGGGPKKLGGGAPAVGGGPKAMGSSLDDEHILSALLQSDDELVGEDSDSEISDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTGATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRMYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPNEVPGTSDDSTEEPVMKKRTYCTYCPSKIRRKANASCKKCKKVICREHNIDMCQSCF

SEQ ID NO: 13 - DNA, 1804 bp, Artificial Sequence
  haPB
  CDS (12)..(1796)
accggtccggcatgggatcttctctggacgacgagcacatcctgtctgccctgctgcagtctgacgatgaactcgtgggcgaagattccgactccgaggtgtccgaccatgtgtctgaggacgacgtgcagtccgataccgaggaagccttcatcgacgaggtgcacgaagtgcagcctacctcttccggctctgagatcctggacgagcagaacgtgatcgagcagcctggatcttccctggcctccaacagaatcctgacactgcctcagcggaccatccggggcaagaacaagcactgctggtccacctctaagagcacccggcggtctagagtgtccgctctgaatattgtgcggtcccagaggggccccaccagaatgtgccggaacatctacgaccctctgctgtgcttcaagctgttcttcaccgacgagatcatctccgagatcgtgaagtggaccaacgccgagatctctctgaagcggcgcgagtctatgacctctgccaccttccgggacaccaacgaggatgagatctacgccttcttcggcatcctggtcatgacagccgtgcggaaggacaaccacatgtccaccgacgacctgttcgacagatccctgtccatggtgtacgtgtccgtgatgtccagggacagattcgacttcctgatccggtgcctgcggatggacgacaagtctatcagacccacactgcgcgagaacgacgtgttcacacctgtgcggaagatctgggacctgttcatccaccagtgcatccagaactacacccctggcgctcacctgaccatcgacgaacagctgctgggcttcagaggcagatgccctttccgggtgtacatccccaacaagccctctaagtacggcatcaagatcctgatgatgtgcgactccggcaccaagtacatgatcaacggcatgccctacctcggcagaggcacccaaacaaatggcgtgccactgggcgagtactacgtgaaagaactgtccaagcctgtgcacggctcctgcagaaacatcacctgtgataactggttcacctccattcctctggccaagaacctgctgcaagagccttacaagctgacaatcgtgggcaccgtgcggtccaacaagcgggaaattcctgaggtgctgaagaactctcggtccagacctgtgggcacctccatgttctgtttcgacggccctctgacactggtgtcctacaagcctaagcctgccaagatggtgtacctgctgtcctcctgtgacgaggacgccagcatcaatgagtccaccggcaagccccagatggtcatgtactacaaccagaccaaaggcggcgtggacaccctggaccagatgtgctctgtgatgacctgctccagaaagaccaacagatggcccatggctctgctgtacggcatgatcaatatcgcctgcatcaacagcttcatcatctactcccacaacgtgtcctccaagggcgagaaggtgcagtcccggaaaaagttcatgcggaacctgtatatgtccctgacctccagcttcatgagaaagcggctggaagcccctacactgaagcgctacctgcgggacaacatctccaacatcctgcctaaagaggtgcccggcaccagcgacgactctacagaggaacccgtgatgaagaagaggacctactgcacctactgtccctccaagatccggcggaaggccaacgcctcttgcaaaaagtgcaagaaagtgatctgccgcgagcacaacatcgatatgtgccagtcctgcttctgagcggccgc

SEQ ID NO: 14 - protein, 594 aa, Artificial Sequence
  Synthetic Construct
MGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 15 - DNA, 2545 bp, Artificial Sequence
  KAT2A-haPB-Taf3
  CDS (15)..(2537)
ccggtggatccggcatgaaggaaaagggcaaagagctgaaggaccccgaccagctgtacaccacactgaagaatctgctggcccagatcaagtctcacccctccgcctggcctttcatggaacccgtgaagaagtctgaggcccctgactactacgaagtgatcagattccccatcgacctcaagaccatgaccgagcggctgagatcccggtactacgtgaccagaaagctgttcgtggccgacctgcagagagtgatcgccaactgtagagagtacaaccctcctgactccgagtactgcagatgcgcctccgctctggaaaagttcttctacttcaagctgaaagaaggcggcctgatcgacaagaagcttggaggcggagcaccagctgttggcggaggacctaaaaaactcggaggtggcgctcctgctgtcggaggcggacctaaagctatgggcagctctctggacgacgagcacatcctgtctgccctgctgcagtccgacgatgaactagtgggcgaagattccgactccgaggtgtccgaccatgtgtctgaggacgacgtgcagtccgataccgaggaagccttcatcgacgaggtgcacgaagtgcagcctacctcttccggctctgagatcctggacgagcagaacgtgatcgagcagcctggatcttccctggcctccaacagaatcctgacactgcctcagcggaccatccggggcaagaacaagcactgctggtccacctctaagagcacccggcggtctagagtgtccgctctgaatattgtgcggtcccagaggggccccaccagaatgtgccggaacatctacgaccctctgctgtgcttcaagctgttcttcaccgacgagatcatctccgagatcgtgaagtggaccaacgccgagatctctctgaagcggcgcgagtctatgacctctgccaccttccgggacaccaacgaggatgagatctacgccttcttcggcatcctggtcatgacagccgtgcggaaggacaaccacatgtccaccgacgacctgttcgacagatccctgtccatggtgtacgtgtccgtgatgtccagggacagattcgacttcctgatccggtgcctgcggatggacgacaagtctatcagacccacactgcgcgagaacgacgtgttcacacctgtgcggaagatctgggacctgttcatccaccagtgcatccagaactacacccctggcgctcacctgaccatcgacgaacagctgctgggcttcagaggcagatgccctttccgggtgtacatccccaacaagccctctaagtacggcatcaagatcctgatgatgtgcgactccggcaccaagtacatgatcaacggcatgccctacctcggcagaggcacccaaacaaatggcgtgccactgggcgagtactacgtgaaagaactgtccaagcctgtgcacggctcctgcagaaacatcacctgtgataactggttcacctccattcctctggccaagaacctgctgcaagagccttacaagctgacaatcgtgggcaccgtgcggtccaacaagcgggaaattcctgaggtgctgaagaactctcggtccagacctgtgggcacctccatgttctgtttcgacggccctctgacactggtgtcctacaagcctaagcctgccaagatggtgtacctgctgtcctcctgtgacgaggacgccagcatcaatgagtccaccggcaagccccagatggtcatgtactacaaccagaccaaaggcggcgtggacaccctggaccagatgtgctctgtgatgacctgctccagaaagaccaacagatggcccatggctctgctgtacggcatgatcaatatcgcctgcatcaacagcttcatcatctactcccacaacgtgtcctccaagggcgagaaggtgcagtcccggaaaaagttcatgcggaacctgtatatgtccctgacctccagcttcatgagaaagcggctggaagcccctacactgaagcgctacctgcgggacaacatctccaacatcctgcctaaagaggtgcccggcaccagcgacgactctacagaggaacccgtgatgaagaagaggacctactgcacctactgtccctccaagatccggcggaaggccaacgcctcttgcaaaaagtgcaagaaagtgatctgccgcgagcacaacatcgatatgtgccagtcctgcttcgccgctgctaaacttggtggtggcgcgccggcagtcggcggaggtccaaaagctgctgataagggcgctgccgtgatcagagatgagtggggcaatcagatctggatctgtcctggctgcaacaagcctgacgacggctctcctatgatcggctgcgacgactgtgacgattggtatcactggccctgcgtgggcatcatgaccgctccacctgaagaaatgcagtggttctgccccaagtgcgccaacaagaagaaggataagaagcacaagaagcgcaagcacagggcccactgatgagcggccgc

SEQ ID NO: 16 - protein, 839 aa, Artificial Sequence
  Synthetic Construct
MKEKGKELKDPDQLYTTLKNLLAQIKSHPSAWPFMEPVKKSEAPDYYEVIRFPIDLKTMTERLRSRYYVTRKLFVADLQRVIANCREYNPPDSEYCRCASALEKFFYFKLKEGGLIDKKLGGGAPAVGGGPKKLGGGAPAVGGGPKAMGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCFAAAKLGGGAPAVGGGPKAADKGAAVIRDEWGNQIWICPGCNKPDDGSPMIGCDDCDDWYHWPCVGIMTAPPEEMQWFCPKCANKKKDKKHKKRKHRAH

SEQ ID NO: 17 - DNA, 101 bp, Artificial Sequence
  primer
  primer (1)..(101)
tagtagctagcttaaccctagaaagataatcatattgtgacgtacgttaaagataatcatgcgtaaaattgacgcatgtcgacgagcgtcacagcacaacc

SEQ ID NO: 18 - DNA, 72 bp, Artificial Sequence
  primer
  primer (1)..(72)
tagtacatatgttaaccctagaaagatagtctgcgtaaaattgacgcatggtgcactctcagtacaatctgc

SEQ ID NO: 19 - DNA, 43 bp, Artificial Sequence
  primer
  primer (1)..(43)
atcgtggcctcggtggcctgaattccctagaaagatagtctgc

SEQ ID NO: 20 - protein, 136 aa, Homo sapiens
VIRDEWGNQIWICPGCNKPDDGSPMIGCDDCDDWYHWPCVGIMTAPPEEMQWFCPKCANKKKDKKHKKRKHRAHKLGGGAPAVGGGPKKLGGGAPAVGGGPKAMGSSLDDEHILSALLQSDDELVGEDSDSEISDH

SEQ ID NO: 21 - protein, 117 aa, Homo sapiens
KEKGKELKDPDQLYTTLKNLLAQIKSHPSAWPFMEPVKKSEAPDYYEVIRFPIDLKTMTERLRSRYYVTRKLFVADLQRVIANCREYNPPDSEYCRCASALEKFFYFKLKEGGLIDK

SEQ ID NO: 22 - protein, 28 aa, Artificial Sequence
  peptide
KLGGGAPAVGGGPKKLGGGAPAVGGGPK

SEQ ID NO: 23 - protein, 24 aa, Artificial Sequence
  peptide
AAAKLGGGAPAVGGGPKAADKGAA

SEQ ID NO: 24 - DNA, 67 bp, Trichoplusia ni
ttaaccctagaaagataatcatattgtgacgtacgttaaagataatcatgcgtaaaattgacgcatg

SEQ ID NO: 25 - DNA, 39 bp, Trichoplusia ni
catgcgtcaattttacgcagactatctttctagggttaa

SEQ ID NO: 26 - DNA, 20 bp, Artificial Sequence
  Primer
tattggtagcccacaagctg

SEQ ID NO: 27 - DNA, 25 bp, Artificial Sequence
  Primer
tttctttcagtgctatgttatggtg

SEQ ID NO: 28 - DNA, 17 bp, Artificial Sequence
  Primer
ggttgtgctgtgacgct

SEQ ID NO: 29 - DNA, 2249 bp, Artificial Sequence
  Kat2a-haPB
  CDS (19)..(2241)
accggtggatccggcatgaaggaaaagggcaaagagctgaaggaccccgaccagctgtacaccacactgaagaatctgctggcccagatcaagtctcacccctccgcctggcctttcatggaacccgtgaagaagtctgaggcccctgactactacgaagtgatcagattccccatcgacctcaagaccatgaccgagcggctgagatcccggtactacgtgaccagaaagctgttcgtggccgacctgcagagagtgatcgccaactgtagagagtacaaccctcctgactccgagtactgcagatgcgcctccgctctggaaaagttcttctacttcaagctgaaagaaggcggcctgatcgacaagaagcttggaggcggagcaccagctgttggcggaggacctaaaaaactcggaggtggcgctcctgctgtcggaggcggacctaaagctatgggcagctctctggacgacgagcacatcctgtctgccctgctgcagtccgacgatgaactagtgggcgaagattccgactccgaggtgtccgaccatgtgtctgaggacgacgtgcagtccgataccgaggaagccttcatcgacgaggtgcacgaagtgcagcctacctcttccggctctgagatcctggacgagcagaacgtgatcgagcagcctggatcttccctggcctccaacagaatcctgacactgcctcagcggaccatccggggcaagaacaagcactgctggtccacctctaagagcacccggcggtctagagtgtccgctctgaatattgtgcggtcccagaggggccccaccagaatgtgccggaacatctacgaccctctgctgtgcttcaagctgttcttcaccgacgagatcatctccgagatcgtgaagtggaccaacgccgagatctctctgaagcggcgcgagtctatgacctctgccaccttccgggacaccaacgaggatgagatctacgccttcttcggcatcctggtcatgacagccgtgcggaaggacaaccacatgtccaccgacgacctgttcgacagatccctgtccatggtgtacgtgtccgtgatgtccagggacagattcgacttcctgatccggtgcctgcggatggacgacaagtctatcagacccacactgcgcgagaacgacgtgttcacacctgtgcggaagatctgggacctgttcatccaccagtgcatccagaactacacccctggcgctcacctgaccatcgacgaacagctgctgggcttcagaggcagatgccctttccgggtgtacatccccaacaagccctctaagtacggcatcaagatcctgatgatgtgcgactccggcaccaagtacatgatcaacggcatgccctacctcggcagaggcacccaaacaaatggcgtgccactgggcgagtactacgtgaaagaactgtccaagcctgtgcacggctcctgcagaaacatcacctgtgataactggttcacctccattcctctggccaagaacctgctgcaagagccttacaagctgacaatcgtgggcaccgtgcggtccaacaagcgggaaattcctgaggtgctgaagaactctcggtccagacctgtgggcacctccatgttctgtttcgacggccctctgacactggtgtcctacaagcctaagcctgccaagatggtgtacctgctgtcctcctgtgacgaggacgccagcatcaatgagtccaccggcaagccccagatggtcatgtactacaaccagaccaaaggcggcgtggacaccctggaccagatgtgctctgtgatgacctgctccagaaagaccaacagatggcccatggctctgctgtacggcatgatcaatatcgcctgcatcaacagcttcatcatctactcccacaacgtgtcctccaagggcgagaaggtgcagtcccggaaaaagttcatgcggaacctgtatatgtccctgacctccagcttcatgagaaagcggctggaagcccctacactgaagcgctacctgcgggacaacatctccaacatcctgcctaaagaggtgcccggcaccagcgacgactctacagaggaacccgtgatgaagaagaggacctactgcacctactgtccctccaagatccggcggaaggccaacgcctcttgcaaaaagtgcaagaaagtgatctgccgcgagcacaacatcgatatgtgccagtcctgcttctgagcggccgc

SEQ ID NO: 30 - protein, 740 aa, Artificial Sequence
  Synthetic Construct
KEKGKELKDPDQLYTTLKNLLAQIKSHPSAWPFMEPVKKSEAPDYYEVIRFPIDLKTMTERLRSRYYVTRKLFVADLQRVIANCREYNPPDSEYCRCASALEKFFYFKLKEGGLIDKKLGGGAPAVGGGPKKLGGGAPAVGGGPKAMGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 31 - DNA, 2101 bp, Artificial Sequence
  haPB-Taf3
  CDS (12)..(2090)
accggtccggcatgggatcttctctggacgacgagcacatcctgtctgccctgctgcagtctgacgatgaactcgtgggcgaagattccgactccgaggtgtccgaccatgtgtctgaggacgacgtgcagtccgataccgaggaagccttcatcgacgaggtgcacgaagtgcagcctacctcttccggctctgagatcctggacgagcagaacgtgatcgagcagcctggatcttccctggcctccaacagaatcctgacactgcctcagcggaccatccggggcaagaacaagcactgctggtccacctctaagagcacccggcggtctagagtgtccgctctgaatattgtgcggtcccagaggggccccaccagaatgtgccggaacatctacgaccctctgctgtgcttcaagctgttcttcaccgacgagatcatctccgagatcgtgaagtggaccaacgccgagatctctctgaagcggcgcgagtctatgacctctgccaccttccgggacaccaacgaggatgagatctacgccttcttcggcatcctggtcatgacagccgtgcggaaggacaaccacatgtccaccgacgacctgttcgacagatccctgtccatggtgtacgtgtccgtgatgtccagggacagattcgacttcctgatccggtgcctgcggatggacgacaagtctatcagacccacactgcgcgagaacgacgtgttcacacctgtgcggaagatctgggacctgttcatccaccagtgcatccagaactacacccctggcgctcacctgaccatcgacgaacagctgctgggcttcagaggcagatgccctttccgggtgtacatccccaacaagccctctaagtacggcatcaagatcctgatgatgtgcgactccggcaccaagtacatgatcaacggcatgccctacctcggcagaggcacccaaacaaatggcgtgccactgggcgagtactacgtgaaagaactgtccaagcctgtgcacggctcctgcagaaacatcacctgtgataactggttcacctccattcctctggccaagaacctgctgcaagagccttacaagctgacaatcgtgggcaccgtgcggtccaacaagcgggaaattcctgaggtgctgaagaactctcggtccagacctgtgggcacctccatgttctgtttcgacggccctctgacactggtgtcctacaagcctaagcctgccaagatggtgtacctgctgtcctcctgtgacgaggacgccagcatcaatgagtccaccggcaagccccagatggtcatgtactacaaccagaccaaaggcggcgtggacaccctggaccagatgtgctctgtgatgacctgctccagaaagaccaacagatggcccatggctctgctgtacggcatgatcaatatcgcctgcatcaacagcttcatcatctactcccacaacgtgtcctccaagggcgagaaggtgcagtcccggaaaaagttcatgcggaacctgtatatgtccctgacctccagcttcatgagaaagcggctggaagcccctacactgaagcgctacctgcgggacaacatctccaacatcctgcctaaagaggtgcccggcaccagcgacgactctacagaggaacccgtgatgaagaagaggacctactgcacctactgtccctccaagatccggcggaaggccaacgcctcttgcaaaaagtgcaagaaagtgatctgccgcgagcacaacatcgatatgtgccagtcctgcttcgccgctgctaaacttggtggtggcgcgccggcagtcggcggaggtccaaaagctgctgataagggcgctgccgtgatcagagatgagtggggcaatcagatctggatctgtcctggctgcaacaagcctgacgacggctctcctatgatcggctgcgacgactgtgacgattggtatcactggccctgcgtgggcatcatgaccgctccacctgaagaaatgcagtggttctgccccaagtgcgccaacaagaagaaggataagaagcacaagaagcgcaagcacagggcccactgatgagcggccgc

SEQ ID NO: 32 - protein, 692 aa, Artificial Sequence
  Synthetic Construct
MGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCFAAAKLGGGAPAVGGGPKAADKGAAVIRDEWGNQIWICPGCNKPDDGSPMIGCDDCDDWYHWPCVGIMTAPPEEMQWFCPKCANKKKDKKHKKRKHRAH

SEQ ID NO: 33 - DNA, 23 bp, Artificial Sequence
  Primer
taagagcaccaactgctcttcca

SEQ ID NO: 34 - DNA, 22 bp, Artificial Sequence
  Primer
accagaagagggcaccagatct